(19) United States
(12) Patent Application Publication
(10) Pub. No.: US 2025/0367624 A1
(43) Pub. Date: Dec. 4, 2025

(54) BIOPOLYMER SYNTHESIS BY METAL-POLYMERIC PARTICLES

(71) Applicant: Lenova (Singapore) Pte. Ltd., Singapore (SG)

(72) Inventors: Cyro von Zuben de Valega Negrão, Butanta (BR); Bruno Nobuya Katayama Gobara, Butanta (BR); Ianca Rosa Dias, Butanta (BR); Luiz Henrique Mesquita Souza, Butanta (BR); Bruno Marinaro Verona, Butantã (BR); Natalia Neto Pereira Cerize, Butanta (BR)

(21) Appl. No.: 18/679,003

(22) Filed: May 30, 2024

(51) Int. Cl. C12P 19/34

(52) U.S. Cl. CPC B01J 8/0095; B82Y 5/00; C12P 19/34

(57) ABSTRACT

Various aspects disclosed relate to producing framework compounds that can be used to synthesize oligonucleotides. The framework compounds can include metallic particles coated with a polymeric layer. Initiator molecules are disposed on the polymeric layer and nucleotide building blocks can be added to the initiator molecules using an enzymatic nucleic acid synthesis process to produce a number of oligonucleotides.

### BACKGROUND

Polymers can be synthesized by the addition of monomer units to form macromolecules having a number of repeating subunits. Polymers can be formed through synthetic processes and through natural processes. In one or more examples, biopolymers can be formed within an organism through a number of biochemical reactions. In some cases, biopolymers can be synthesized outside of an organism via at least one of one or more chemical processes, one or more enzymatic processes, or one or more electrochemical processes.

### SUMMARY OF THE DISCLOSURE

One or more aspects disclosed relate to an article that comprises a framework compound that can be used to form oligonucleotides. The framework compound can include a metallic particle and a polymeric layer disposed on the metallic particle. The polymeric layer can be comprised of one or more polymeric materials. The framework molecule can also include a plurality of initiator molecules disposed on the polymeric layer. The plurality of initiator molecules can comprise a plurality of nucleotides. Additionally, the metallic particle can be bound to the polymeric layer by first electrostatic interactions between the metallic particle and one or more first functional groups of the one or more polymeric materials. Further, the plurality of initiator molecules can be bound to the polymeric layer by second electrostatic interactions between one or more second functional groups of the plurality of initiator molecules and the one or more first functional groups of the one or more polymeric materials.

In addition, one or more aspects disclosed relate to a method of forming a framework compound that can be used to form oligonucleotides. The method includes combining an amount of metallic particles with an amount of one or more polymeric materials in one or more containers with an amount of a polar solvent to form coated metallic particles. Individual coated metallic particles can comprise a polymeric layer of the one or more polymeric materials disposed on a metallic particle and the polymeric layer can be bound to the metallic particle by first electrostatic interactions. The method can also include combining the coated metallic particles with a plurality of initiator molecules in the one or more containers to form a plurality of framework compounds. The plurality of initiator molecules can comprise a plurality of nucleotides and the plurality of initiator molecules can be bound to the coated metallic particles by second electrostatic interactions.

Further, one or more aspects disclosed relate to a method of producing oligonucleotides using a framework compound comprised of a metal-polymer hybrid particle. The method can include providing a plurality of framework compounds in a polar solvent disposed in one or more containers. The plurality of framework compounds can comprise a number of coated metallic particles having a plurality of initiator molecules bound to the number of coated metallic particles. Individual coated metallic particles can comprise a metallic particle coated with a polymeric layer that includes one or more polymeric materials. The method can also include adding a number of nucleotides to individual initiator molecules of the plurality of initiator molecules to produce a plurality of oligonucleotides bound to the polymeric layer. Additionally, the method can include adding a rinsing solution including an amount of a surfactant to the one or more containers to separate the oligonucleotides from the polymeric layer.

### BRIEF DESCRIPTION OF THE FIGURES

The drawings illustrate generally, by way of example, but not by way of limitation, various aspects of the present invention.

FIG. 1 is a diagram showing a process to produce metal-polymer framework compounds for forming oligonucleotides, in accordance with one or more implementations.

FIG. 2 is a diagram showing a process to produce oligonucleotides using metal-polymer framework compounds, in accordance with one or more implementations.

FIG. 3 is diagram of an architecture to encode and decode data using oligonucleotides synthesized using metal-polymer framework compounds, in accordance with one or more implementations.

FIG. 4 is a flow diagram of an example method to make metal-polymer framework compounds for the production of oligonucleotides, in accordance with one or more implementations.

FIG. 5 is a flow diagram of an example method to synthesize oligonucleotides using metal-polymer framework compounds, in accordance with one or more implementations.

FIG. 6 includes an image of an agarose gel after performing an electrophoresis gel representing a number of combinations of materials used to produce framework compounds according to implementations described herein.

FIG. 7 includes an image of an agarose gel after performing an electrophoresis gel representing a number of combinations of materials used to synthesize oligonucleotides with framework compounds according to implementations described herein.

FIG. 8 shows zeta potential measurements for different combinations of amounts of a polymeric material and metallic particles.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the inventive subject matter, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range.

The terms “polynucleotide”, “nucleic acid”, “nucleic acid molecule”, “polynucleotide molecule”, or “oligonucleotide” refer to a linear polymer of nucleotides or nucleosides joined by internucleosidic linkages. A polynucleotide can comprise at least three nucleotides or three nucleosides. Oligonucleotides often range in size from a few monomeric units, e.g., 3-4, to hundreds of monomeric units. Whenever a polynucleotide is represented by a sequence of letters, such as “ATGCCTG,” it will be understood that the nucleotides are in 5′□3′ order from left to right and that in the case of DNA, “A” denotes adenosine or deoxyadenosine, “C” denotes cytosine or deoxycytidine, “G” denotes guanine or deoxyguanosine, and “T” denotes thymine or deoxythymidine, unless otherwise noted. The letters A, C, G, and T may be used to refer to the bases themselves, to nucleosides, or to nucleotides comprising the bases, as is standard in the art.

As used herein, “deoxyribonucleic acid” or “DNA” refers to a natural or modified polynucleotide which has a hydrogen group at the 2′-position of the sugar moiety. DNA can include a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA can include a chain of nucleotides comprising four types of nucleotides: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data”, “nucleic acid sequencing information”, “sequence information”, “nucleic acid sequence”, “nucleotide sequence”, “sequencing read”, or “nucleic acid sequencing read” denotes any information or data that is indicative of the order and identity of the nucleotide bases (e.g., adenine, guanine, cytosine, and thymine or uracil) in a molecule (e.g., oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

The terms, “binary data”, “digital information”, or “digital data” refers to data encoded using the standard binary code, or a base 2 {0,1} alphabet, data encoded using a hexadecimal base 16 alphabet, data encoded using the base 10 {0-9} alphabet, data encoded using ASCII characters, or data encoded using any other discrete alphabet of symbols or characters in a linear encoding fashion.

As used herein, the term “aqueous solution” can refer to a liquid solution that primarily comprises water. For example, an aqueous solution can comprise at least about 50% by weight H₂O, at least about 55% by weight H₂O, at least about 60% by weight H₂O, at least about 65% by weight H₂O, at least about 70% by weight H₂O, at least about 75% by weight H₂O at least about 80% by weight H₂O, at least about 85% by weight H₂O, at least about 90% by weight H₂O, at least about 95% by weight H₂O, or at least about 99% by weight H₂O.

In one or more examples, the synthetic production of biopolymers can take place by joining monomer units in an enzymatic process. Enzymatic synthesis of oligonucleotides can avoid the use of harsh solvents that are typically applied in phosphoramidite oligonucleotide synthesis. Instead, enzymatic synthesis of oligonucleotides is performed in aqueous environments using one or more enzymes that function to add nucleotides to an oligonucleotide chain.

In one or more examples, polynucleotide phosphorylase (PNPase) can be used to produce single stranded nucleic acid molecules by adding single nucleotides to a growing chain of nucleotides. Enzymatic processes based on PNPase to produce oligonucleotides can add modified nucleoside diphosphates to an oligonucleotide chain. The modified nucleoside diphosphates can have 3′ blocking groups that enable the addition of nucleotides to an oligonucleotide chain. Additionally, oligonucleotides can be synthesized with T4 RNA ligase (T4Rnl) using modified nucleoside diphosphates with a 3′ blocking group that are different from the modified nucleoside diphosphates used in PNPase synthesis processes to add nucleotides to an oligonucleotide chain.

DNA polymerases can also be used to enzymatically form oligonucleotides. To illustrate, terminal deoxynucleotidyl transferase (TdT) can be used to add nucleotides to an oligonucleotide chain. TdT can produce homopolymers in an oligonucleotide chain. That is, in a given cycle of nucleotide addition, enzymatic processes using TdT can add multiple instances of a single nucleotide to a growing oligonucleotide chain. In various examples, in a single cycle to add nucleotides to an oligonucleotide chain, synthesis of oligonucleotides using TdT can result in a number of adenine molecules being added to the oligonucleotide chain, a number of thymine molecules being added to the oligonucleotide chain, a number of guanine molecules being added to the oligonucleotide chain, a number of cytosine molecules being added to the oligonucleotide chain, or a number of uracil molecules being added to the oligonucleotide chain.

In one or more illustrative examples, nucleic acids can be synthesized by adding nucleotides to a molecular scaffold that comprises an intermediate oligonucleotide chain. For example, deoxyribonucleic acid (DNA) molecules and ribonucleic acid (RNA) molecules can be formed by coupling monomer units comprised of adenine (A), guanine (G), cytosine (C), and thymine (T), in the case of DNA, or A, G, C, and uracil (U), in the case of RNA. Typically, synthetic polynucleotides are produced according to a number of predetermined sequences. The predetermined sequences can correspond to at least one of the primers used in polynucleotide sequencing operations. The predetermined sequences can also correspond to identifiers that can be used to identify molecules and/or families of molecules after the sequencing process has been performed. In various examples, the predetermined sequences can correspond to digital data that has been encoded within sequences of oligonucleotides.

In at least some examples, the coupling of nucleotides can include successively adding nucleotides to an intermediate oligonucleotide chain until a completed oligonucleotide is produced having a sequence of bases that corresponds to the predetermined sequence. In various implementations, framework compounds can be used to synthesize oligonucleotides. The framework compounds can include a metallic particle and a polymeric layer disposed on the metallic particle. In one or more examples, the metallic particle can have magnetic properties. For example, the metallic particle can include a superparamagnetic metal. Additionally, the polymeric layer can be comprised of one or more polymeric materials that form positively charged ions in a polar solution. The framework compounds can also include initiator molecules disposed on the polymeric layer.

The metallic particle and the polymeric layer can be bound by non-covalent interactions between the metallic particle and one or more functional groups of polymeric materials included in the polymeric layer. In this way, electrons are not shared between the metallic particle and one or more functional groups of polymeric materials included in the polymeric layer. For example, the metallic particle and the polymeric layer can be bound by electrostatic interactions. Further, the initiators can be bound to the polymeric layer by electrostatic interactions. The electrostatic interactions that bind the metallic particles to the molecules of the polymeric layer and that bind the initiator molecules to the molecules of the polymeric layer can include at least one of ionic interactions or Van der Waals forces.

In at least some examples, the non-covalent interactions can be at least one of identified or characterized by implementing Fourier Transform Infrared Spectroscopy (FTIR). In one or more additional examples, the non-covalent interactions can be at least one of identified or characterized by implementing one or more dynamic light scattering techniques. In various examples, the non-covalent interactions can be at least one of identified or characterized by measuring zeta potential using one or more dynamic light scattering techniques. Further, the non-covalent interactions can be identified and/or characterized by the analysis of particle size using one or more dynamic light scattering techniques. In still other examples, the non-covalent interactions can be at least one of identified or characterized by implementing one or more thermogravimetric analysis (TGA) techniques.

In one or more examples, the electrostatic interactions can include interactions between molecules having one or more oppositely charged functional groups. In at least some examples, in a polar solution, the metallic particles can be negatively charged and molecules comprising the one or more polymeric materials can be positively charged. In this way, the metallic particles and the molecules comprising the polymeric materials can be attracted to each other and form electrostatic interactions, such as ionic bonds. Further, the initiator molecules can have a negative charge in a polar solution. As a result, the initiator molecules can also form electrostatic interactions with the molecules comprising the polymeric materials. The electrostatic interactions between the metallic particles, the one or more polymeric materials, and the initiator molecules can produce a metal-polymer hybrid framework compound that can be used to synthesize oligonucleotides.

The initiator molecules disposed on the polymeric layer can be used as initial sequences for producing oligonucleotides. In various examples, enzymatic nucleic acid synthesis techniques can be used to add nucleotides to the initiator molecules to produce oligonucleotides having predetermined nucleic acid sequences. In one or more examples, one or more instances of a given nucleotide can be added to an intermediate oligonucleotide chain in an individual nucleotide addition cycle according to the predetermined sequences. In one or more additional examples, after completion of a number of cycles of adding nucleotides to the initiator molecules, the oligonucleotides can be removed from the polymeric layer. In at least some cases, the predetermined nucleic acid sequences can encode one or more segments of digital data.

By producing metal-polymer framework compounds that include components that are bound via electrostatic interactions, oligonucleotides can be produced without the use of harsh and/or harmful solvents. For example, in oligonucleotide synthesis using at least one of chemical processes or electrochemical processes, various acidic solutions and/or other harsh or toxic solvents are used in molecule separation processes. To illustrate, harmful solvents are typically used during cycles to add nucleotides to a growing oligonucleotide chain and to separate completed oligonucleotides from framework compounds or framework substrates because covalent bonds are formed between the molecules taking part in the oligonucleotide synthesis processes. The strong solvents are used to break the covalent bonds formed between these compounds. In contrast, the techniques described herein can use aqueous solutions and mild surfactants to separate completed oligonucleotides from the framework compounds because the electrostatic interactions between molecules taking part in the processes described are not as strong as the covalent bonds used in typical chemical and electrochemical oligonucleotide synthesis processes.

Additionally, enzymatic oligonucleotide synthesis processes can produce oligonucleotides having a greater length than oligonucleotides generated using typical phosphoramidite processes. For example, the length of oligonucleotides produced using phosphoramidite processes is rarely up to 200 nucleotides in length. In contrast, synthesis of oligonucleotides using enzymatic processes produces oligonucleotides having lengths from at least 400 nucleotides or 500 nucleotides up to 1000 nucleotides or more. In situations where the oligonucleotides are used to encode digital data, the ability to synthesize oligonucleotides with lengths that are longer than typical phosphoramidite processes can result in more data being encoded in individual oligonucleotides. As a result, when the data encoded by the oligonucleotides is decoded, fewer oligonucleotides are retrieved and fewer sequencing operations are used to determine the sequences of the retrieved oligonucleotides, which leads to fewer materials and resources being used in the retrieval of data stored by oligonucleotides generated using implementations described herein.

Further, the enzymatic synthesis of oligonucleotides according to implementations described herein can be performed faster than phosphoramidite synthesis of oligonucleotides because fewer operations are performed in enzymatic synthesis of oligonucleotides in relation to phosphoramidite oligonucleotide synthesis. For instance, the enzymatic oligonucleotide synthesis operations described herein do not involve the blocking and deblocking operations performed at each cycle of adding oligonucleotides to an oligonucleotide chain in phosphoramidite oligonucleotide synthesis.

In still other examples, by producing oligonucleotides through enzymatic processes that produce homopolymers during each cycle of adding nucleotides, the accuracy of data retrieval processes is increased in relation to the accuracy of data retrieval processes based on data encoded by oligonucleotides synthesized by chemical processes and/or electrochemical processes. For example, homopolymers produced during an enzymatic oligonucleotide synthesis process correspond to a single nucleotide in a sequence used to encoded digital data. Thus, during the decoding process the presence of one or more nucleotides at a given group of positions of the oligonucleotide can correspond to a single nucleotide in the data encoding sequence.

In one or more examples, in typical oligonucleotide synthesis processes a single nucleotide is added to a growing oligonucleotide chain in each cycle. Errors can occur in typical oligonucleotide synthesis processes when one or more nucleotides are omitted from a growing oligonucleotide chain during one or more nucleotide addition cycles due to, for example, one or more chemical reactions not taking place in a reaction vessel. This can result in one or more nucleotides being missing from an oligonucleotide sequence. In addition to these types of errors, one or more errors can occur during sequencing operations, such as during enrichment operations and/or amplification operations. In situations where oligonucleotides are comprised of single nucleotides corresponding to a predetermined sequence, the probability of errors in sequencing operations causing errors in the decoding of the oligonucleotide sequencing data can increase because errors in the oligonucleotide sequences that are caused by erroneous reactions that occurred during sequencing processes can cause the decoded sequences to be less likely to correspond to an encoded sequence.

In scenarios where homopolymers are generated in the synthesis of oligonucleotides, the probability that a missing nucleotide will result in an error in the decoded sequences is minimized because multiple instances of a given nucleotide are present in the oligonucleotide for each nucleotide in an encoded sequence. As a result, the impact of a single missing nucleotide in the oligonucleotide sequence caused by errors in the oligonucleotide synthesis process and/or in one or more sequencing operations is minimized because the oligonucleotide sequence still includes at least one other instance of the missing nucleotide in the sequence. Thus, the decoding of the oligonucleotide sequence can still be performed accurately because at least one instance of the nucleotide present in the encoded oligonucleotide sequence is present in sequencing reads that correspond to the oligonucleotide molecules that have erroneous sequences. Accordingly, the decoding process can result in producing a digital data sequence that corresponds to the originally recorded digital data.

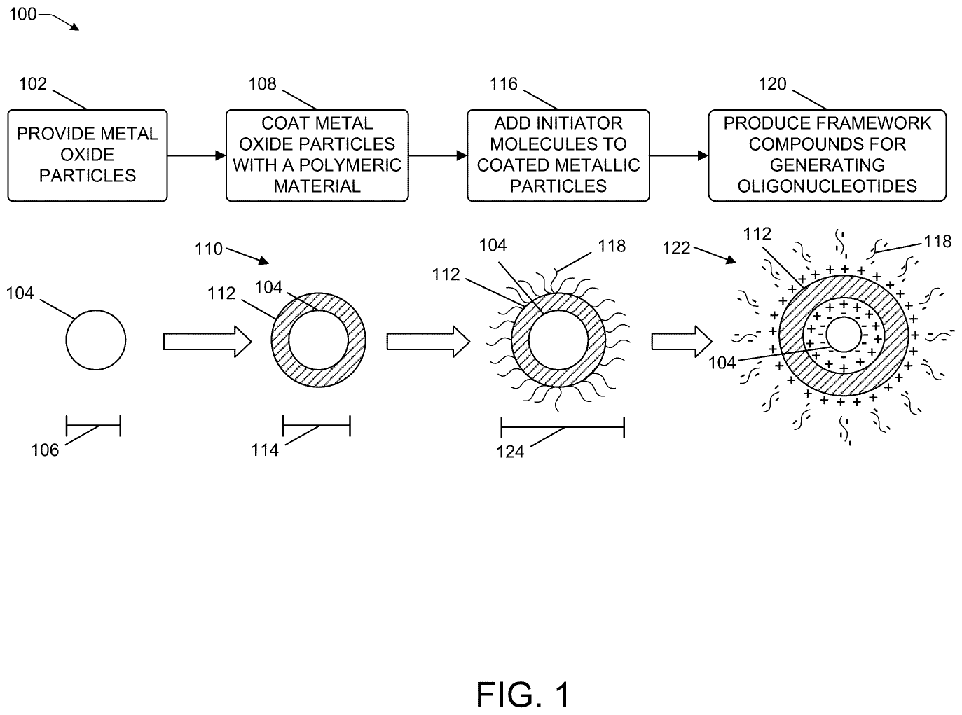

FIG. 1 is a diagram showing a process 100 to produce metal-polymer framework compounds for forming oligonucleotides, in accordance with one or more implementations. The process 100 can include, at 102, providing metallic particles 104. In at least some examples, the metallic particles 104 can include metal oxide particles. The metallic particles 104 can comprise one or more metals that have magnetic properties. In one or more examples, the metallic particles 104 can comprise one or more metals that have superparamagnetic properties. Superparamagnetism can be found in ferromagnetic or ferrimagnetic nanoparticles. In various examples, superparamagnetic materials can randomly flip direction based on temperatures applied to the materials. The average value of magnetization of superparamagnetic materials can be zero or near zero in the absence of an external magnetic field while having a relatively high level of magnetization in the presence of an external magnetic field. In one or more illustrative examples, the metallic particles 104 can comprise one or more transition metals. For example, the metallic particles 104 can comprise oxides of at least one of iron (Fe), cobalt (Co), nickel (Ni), or manganese (Mn). In one or more additional illustrative examples, the metallic particles 104 can comprise Fe₂O₃. In one or more further illustrative examples, the metallic particles 104 can comprise Fe₃O₄.

The metallic particles 104 can have one or more dimensions, such as an example dimension 106. In one or more examples, the metallic particles 104 can have a spherical shape. In these scenarios, the example dimension 106 can comprise a diameter. The metallic particles 104 can individually have an example dimension 106, such as a diameter, of at least about 0.1 nanometers, at least about 0.5 nanometers, at least about 1 nanometer, at least 2 nanometers, at least about 5 nanometers, at least about 10 nanometers, at least about 20 nanometers, at least about 30 nanometers, at least about 40 nanometers, at least about 50 nanometers, at least about 60 nanometers, at least about 70 nanometers, at least about 80 nanometers, at least about 90 nanometers, or at least about 100 nanometers. Additionally, the metallic particles 104 can individually have an example dimension 106 no greater than about 500 nanometers, no greater than about 450 nanometers, no greater than about 400 nanometers, no greater than about 350 nanometers, no greater than about 300 nanometers, no greater than about 250 nanometers, no greater than about 200 nanometers, or no greater than about 150 nanometers. In one or more illustrative examples, the metallic particles 104 can individually have example dimensions 106 from about 0.1 nanometers to about 2 nanometers, from about 0.5 nanometers to about 5nanometers, from about 2 nanometers to about 500 nanometers, from about 10 nanometers to about 400 nanometers, from about 50 nanometers to about 300 nanometers, from about 5 nanometers to about 50 nanometers, from about 50 nanometers to about 150 nanometers, from about 100 nanometers to about 200 nanometers, from about 20 nanometers to about 100 nanometers, or from about 2 nanometers to about 30 nanometers. In various examples, dimensions of the metallic particles 104 can be measured according to one or more dynamic light scattering techniques.

The process 100 can include, at 108, coating the metallic particles 104 with a polymeric material to produce coated metallic particles 110. The coated metallic particles 110 can include a polymeric layer 112 disposed on the metallic particles 104. In one or more examples, the polymeric layer 112 can be comprised of polymeric molecules having a number average molecular weight of at least about 2 kilodaltons (kDa), at least about 5 kDa, at least about 8 kDa, at least about 10 kDa, at least about 12 kDa, at least about 15 kDa, at least about 18 kDa, at least about 20 kDa, or at least about 25 kDa. Additionally, the polymeric layer 112 can be comprised of polymeric molecules having a number average molecular weight of no greater than about 100 kDa, no greater than about 90 kDa, no greater than about 80 kDa, no greater than about 70 kDa, no greater than about 60 kDa, no greater than about 50 kDa, no greater than about 40 kDa, or no greater than about 30 kDa. In one or more illustrative examples, the polymeric layer 112 can be comprised of polymeric molecules having a number average molecular weight from about 2 kDa to about 100 kDa, from about 5 kDa to about 80 kDa, from about 10 kDa to about 60 kDa, from about 5 kDa to about 15 kDa, from about 2 kDa to about 10 kDa, from about 2 kDa to about 20 kDa, from about 10 kDa to about 20 kDa, or from about 15 kDa to about 30 kDa. In one or more additional illustrative examples, the polymeric layer 112 can be comprised of at least one of poly(N,N-dimethylaminoethyl methacrylate), polyethylenimine, poly-L-lysine, polyvinylamine, or polyallylamine.

The metallic particles 104 can be coated with the polymeric material in a solution phase process. For example, an amount of the metallic particles 104 and an amount of the polymeric material can be included in one or more polar solutions and combined in one or more containers. The one or more polar solutions can include one or more polar solvents. In one or more examples, a polar solvent can include molecules having a dipole moment that is formed due to the unequal sharing of electrons between the different atoms of the molecules that comprise the polar solvent. In one or more illustrative examples, the one or more polar solutions can include an aqueous solution. In one or more additional illustrative examples, the one or more polar solutions can include at least one of water, (tris(2-carboxyethyl)phosphine) (TCEP), dimethyl sulfoxide (DMSO), Ethylenediaminetetraacetic acid (EDTA), ethyl acetate, acetic acid, isopropanol, ethanol, or methanol.

In various examples, the metallic particles 104 can comprise first charged particles in the one or more polar solutions and molecules of the polymeric material can comprise second charged particles in the one or more polar solutions. The first charged particles can be oppositely charged than the second charged particles. In one or more examples, the metallic particles 104 can be negatively charged in the one or more polar solutions and molecules of the polymeric material can be positively charged in the one or more polar solutions. In these scenarios, the coated metallic particles 110 can be formed through electrostatic interactions between the negatively charged metallic particles 104 and the positively charged polymeric material molecules. In this way, molecules of the polymeric material are bound to the metallic particles 104 by electrostatic interactions to produce the coated metallic particles 110.

In one or more examples, the polymeric layer 112 can cover at least about 75% of an outer surface of the metallic particles 104, at least about 80% of an outer surface of the metallic particles 104, at least about 85% of an outer surface of the metallic particles 104, at least about 90% of an outer surface of the metallic particles 104, at least about 95% of an outer surface of the metallic particles, or at least about 99% of an outer surface of the metallic particles. In various examples, individual metallic particles 104 can be completely encased by the polymeric layer 112.

The coated metallic particles 110 can have one or more dimensions, such as an additional example dimension 114. In at least some examples, the coated metallic particles 110 can have a spherical shape. In these scenarios, the additional example dimension 114 can include a diameter. In various examples, the additional example dimension 114 can be at least about 0.5 nanometers, at least about 1 nanometer, at least about 2 nanometers, at least 5 about nanometers, at least about 10 nanometers, at least about 20 nanometers, at least about 30 nanometers, at least about 40 nanometers, at least about 50 nanometers, at least about 60 nanometers, at least about 70 nanometers, at least about 80 nanometers, at least about 90 nanometers, at least about 100 nanometers, at least about 120 nanometers, or at least about 150 nanometers. Further, the additional example dimension 114 can be no greater than about 750 nanometers, no greater than about 600 nanometers, no greater than about 500 nanometers, no greater than about 450 nanometers, no greater than about 400 nanometers, no greater than about 350 nanometers, no greater than about 300 nanometers, no greater than about 250 nanometers, or no greater than about 200 nanometers. In one or more illustrative examples, the additional example dimension 114 can be from about 0.5 nanometers to about 5 nanometers, from about 1 nanometer to about 10 nanometers, about 10 nanometers to about 750 nanometers, from about 50 nanometers to about 500 nanometers, from about 100 nanometers to about 300 nanometers, from about 20 nanometers to about 100 nanometers, from about 50 nanometers to about 150 nanometers, from about 100 nanometers to about 200 nanometers, from about 150 nanometers to about 250 nanometers, or from about 200 nanometers to about 300 nanometers. In various examples, dimensions of the coated metallic particles 110 can be measured according to one or more dynamic light scattering techniques.

In one or more examples, the polymeric layer 112 can have a thickness. The thickness of the polymeric layer 112 for one or more individual metallic particles 104 can include a difference between the example dimension 106 and the additional example dimension 114. For example, the thickness of the polymeric layer 112 can include a difference between a diameter of the metallic particles 104 and a diameter of the coated metallic particles 110. In one or more illustrative examples, a thickness of the polymeric layer 112 can be from about 5 nanometers to about 100 nanometers, from about 10 nanometers to about 80 nanometers, from about 20 nanometers to about 50 nanometers, from about 10 nanometers to about 30 nanometers, from about 20 nanometers to about 40 nanometers, from about 10 nanometers to about 20 nanometers, or from about 5 nanometers to about 15 nanometers. In at least some examples, the polymeric layer 112 can have a thickness such that the metallic particles 104 are sufficiently coated with the one or more polymeric materials of the polymeric layer 112. Further, the polymeric layer 112 can have a thickness such that degradation of the magnetic properties of the metallic particles 104 is controlled as to enable the metallic particles 104 of the coated metallic particles 110 to change magnetic states and have at least a threshold amount of magnetization in the presence of a magnetic field. The threshold amount of magnetization can correspond to an amount of magnetization that enables the coated metallic particles to be bound to a surface in the presence of a magnetic field while being subjected to external forces, such as the flow of fluids through one or more containers that comprise an amount of the coated magnetic particles 110. In one or more illustrative examples, the magnetic field can be produced by at least one of one or more permanent magnets, one or more temporary magnets, or an electromagnetic device. In one or more additional examples, the magnetic field can be produced by magnets that are comprised of at least one of a ferrite material or an alnico material. The magnetic field can have values no greater than 0.001 Teslas, no greater than 0.005 Teslas, no greater than 0.01 Teslas, no greater than 0.05 Teslas, no greater than 0.1 Teslas, no greater than 0.5 Teslas, no greater than 1 Tesla, no greater than 5 Teslas, or no greater than 10 Teslas.

In at least some examples, a ratio of a weight of the one or more polymeric materials included in the polymeric layer 112 relative to a weight of metallic particles 104 in the polar solution can be at least about 2:1, at least about 3:1, at least about 4:1, at least about 5:1, at least about 6:1, at least about 7:1, or at least about 8:1. Additionally, a ratio of a weight of the one or more polymeric materials included in the polymeric layer 112 relative to a weight of metallic particles 104 in the one or more polar solutions can be no greater than about 15:1, no greater than about 14:1, no greater than about 13:1, no greater than about 12:1, no greater than about 11:1, no greater than about 10:1, or no greater than about 9:1. In one or more illustrative examples, a ratio of a weight of the one or more polymeric materials included in the polymeric layer 112 relative to a weight of metallic particles 104 in the one or more polar solutions can be from about 2:1 to about 15:1, from about 3:1 to about 12:1, from about 4:1 to about 9:1, from about 2:1 to about 5:1, or from about 3:1 to about 6:1. In still other examples, a concentration of the metallic particles 104 in the one or more polar solutions can be from about 10 milligrams/millimeter (mg/mL) to about 30 mg/mL, from about 12 mg/mL to about 25 mg/mL, from about 15 mg/mL to about 20 mg/mL, from about 10 mg/mL to about 20 mg/mL, or from about 15 mg/mL to about 25 mg/mL. In various examples, the amounts of the metallic particles 104 and polymeric materials are provided such that the metallic particles 104 are encased in the polymeric layer 112, while maintaining the influence of a magnetic field on the metallic particles 104. That is, a thickness of the polymeric layer 112 is controlled such that the thickness is not great enough to block the influence of the magnetic field on the metallic particles 104. Additionally, the amounts of the metallic particles 104 and the polymeric materials are provided such that a polar solution include the metallic particles and the polymeric materials is a substantially homogenous mixture that is stable at room temperature for an extended period of time.

The process 100 can also include, at 116, adding initiator molecules 118 to the coated metallic particles 110. The initiator molecules 118 can be added to the coated metallic particles 110 in a solution phase process. For example, the initiator molecules 118 can be added to one or more polar solutions that include the coated metallic particles 110. Additionally, the initiator molecules 118 can be included in one or more additional polar solutions that are added to one or more polar solutions that include the coated metallic particles 110. In various examples, the one or more additional polar solutions used to add the initiator molecules 118 to the coated metallic particles 110 can have a composition that is the same as or similar to that of the one or more polar solutions in which the metallic particles 104 and the one or more polymeric materials were combined to form the coated metallic particles 110. In one or more additional examples, after formation of the coated metallic particles 110, one or more washing operations and/or one or more rinsing operations can be performed before the initiator molecules 118 are combined with the coated metallic particles 110 in one or more polar solutions. Further, in one or more examples, after the coated metallic particles 110 are formed in one or more polar solutions, the initiator molecules 118 can be added to the same batch of one or more polar solutions without at least one of one or more washing operations or one or more rinsing operations being performed.

In various examples, the initiator molecules 118 can comprise at least about 2 nucleotides, at least about 3 nucleotides, at least about 5 nucleotides, at least about 8 nucleotides, at least about 10 nucleotides, at least about 12 nucleotides, at least about 15 nucleotides, at least about 18 nucleotides, at least about 20 nucleotides, at least about 22 nucleotides, or at least about 25 nucleotides. In one or more additional examples, the initiator molecules 118 can comprise no greater than about 100 nucleotides, no greater than about 90 nucleotides, no greater than about 80 nucleotides, no greater than about 70 nucleotides, no greater than about 60 nucleotides, no greater than about 50 nucleotides, no greater than about 40 nucleotides, or no greater than about 30 nucleotides. In one or more illustrative examples, the initiator molecules 118 can comprise from about 2 nucleotides to about 100 nucleotides, from about 5 nucleotides to about 80 nucleotides, from about 10 nucleotides to about 50 nucleotides, from about 2 nucleotides to about 5 nucleotides, from about 5 nucleotides to about 20 nucleotides, from about 15 nucleotides to about 30 nucleotides, from about 25 nucleotides to about 40 nucleotides, or from about 50 nucleotides to about 100 nucleotides.

In one or more examples, the initiator molecules 118 can have a nucleotide sequence such that oligonucleotides can be synthesized using the initiators. For example, the initiator molecules 118 can have a nucleotide sequence such that additional nucleotides can be added to the initiator molecules according to one or more predetermined oligonucleotide sequences. In at least some examples, individual initiator molecules 118 can have a same or similar nucleotide sequence. In one or more illustrative examples, the initiator molecules 118 include at least a portion of a nucleotide sequence of an M13 phage genome.

A ratio of an amount of moles of initiator molecules 118 in one or more polar solutions relative to an amount of moles of the one or more polymeric materials of the polymeric layer 112 can be at least about 2:1, at least about 3:1, at least about 4:1, at least about 5:1, at least about 6:1, or at least about 7:1. Additionally, a ratio of an amount of moles of initiator molecules 118 in one or more polar solutions relative to an amount of moles of the one or more polymeric materials of the polymeric layer 112 can be no greater than about 12:1, no greater than about 11:1, no greater than about 10:1, no greater than about 9:1, or no greater than about 8:1. In one or more illustrative examples, a ratio of an amount of moles of initiator molecules 118 in one or more polar solutions relative to an amount of moles of the one or more polymeric materials of the polymeric layer 112 can be from about 2:1 to about 12:1, from about 3:1 to about 10:1, from about 4:1 to about 8:1, from about 2:1 to about 5:1, from about 3:1 to about 6:1, or from about 4:1 to about 7:1.

In still other examples, a ratio of a weight, such as in grams, of the one or more polymeric materials of the polymeric layer 112 in a polar solution relative to a weight, such as in grams, of the initiator molecules 118 can be at least about 4:1, at least about 5:1, at least about 6:1, at least about 7:1, at least about 8:1, at least about 9:1, at least about 10:1, at least about 11:1, or at least about 12:1. In one or more further examples, a ratio of a weight of the one or more polymeric materials of the polymeric layer 112 in a polar solution relative to a weight of the initiator molecules 118 can be no greater than about 22:1, no greater than about 21:1, no greater than about 20:1, no greater than about 19:1, no greater than about 18:1, no greater than about 17:1, no greater than about 16:1, no greater than about 15:1, no greater than about 14:1, no greater than about 13:1, or no greater than about 12:1. In one or more illustrative examples, a ratio of a weight of the one or more polymeric materials of the polymeric layer 112 in a polar solution relative to a weight of the initiator molecules 118 can be from about 4:1 to about 22:1, from about 5:1 to about 20:1, from about 8:1 to about 15:1, from about 10:1 to about 18:1, from about 12:1 to about 20:1, or from about 10:1 to about 16:1.

Adding initiator molecules 118 to the coated metallic particles 110 at 116 can cause the process 100 to move to 120 where framework compounds 122 are produced for generating oligonucleotides. The framework compounds 122 can include a metallic core comprised of one or more metallic particles 104, a polymeric layer 112 disposed on the one or more metallic particles 104, and a number of initiator molecules 118 located on the polymeric layer 112. The components of the framework compounds 122 can be bound together by electrostatic forces. For example, the one or more metallic particles 104 and the initiator molecules 118 can be negatively charged in one or more polar solutions and the polymeric materials comprising the polymeric layer 112 can be positively charged in the one or more polar solutions. In this way, the negatively charged metallic particles 104 and the positively charged polymeric materials comprising the polymeric layer 112 can be bound by first electrostatic forces. In addition, the negatively charged functional groups of the initiator molecules 118 and the positively charged polymeric materials comprising the polymeric layer 112 can be bound by second electrostatic forces.

The framework compounds 122 can have one or more dimensions, such as a further example dimension 124. In at least some examples, the framework compounds 122 can have a spherical shape. In these scenarios, the further example dimension 124 can include a diameter. In various examples, the further example dimension 124 can be at least 1 nanometer, at least 2 nanometers, at least 5 nanometers, at least 10 nanometers, at least about 20 nanometers, at least about 40 nanometers, at least about 60 nanometers, at least about 80 nanometers, at least about 100 nanometers, at least about 120 nanometers, at least about 140 nanometers, at least about 160 nanometers, at least about 180 nanometers, at least about 200 nanometers, or at least about 250 nanometers. The further example dimension 124 can also be no greater than about 900 nanometers, no greater than about 800 nanometers, no greater than about 700 nanometers, no greater than about 600 nanometers, no greater than about 500 nanometers, no greater than about 400 nanometers, no greater than about 300 nanometers, or no greater than about 250 nanometers. In one or more illustrative examples, the further example dimension 124 can be from about 1 nanometer to about 900 nanometers, from about 2 nanometers to about 700 nanometers, from about 5 nanometers to about 400 nanometers, from about 10 nanometers to about 120 nanometers, from about 1 nanometers to about 10 nanometers, from about 2 nanometers to about 30 nanometers, from about 5 nanometers to about 40 nanometers, from about 2 nanometers to about 20 nanometers, from about 50 nanometers to about 100 nanometers, from about 50 nanometers to about 150 nanometers, from about 100 nanometers about 200 nanometers, or from about 150 nanometers to about 250 nanometers. In various examples, dimensions of the framework compounds 122 can be measured according to one or more dynamic light scattering techniques.

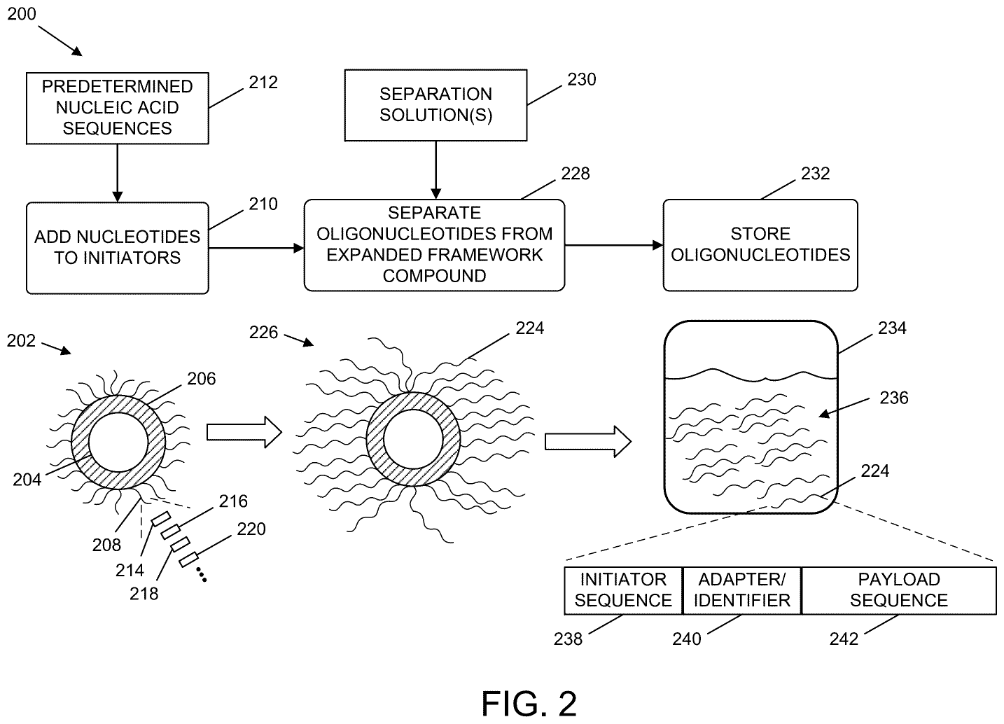

FIG. 2 is a diagram showing process 200 to produce oligonucleotides using metal-polymer framework compounds 202, in accordance with one or more implementations. The framework compounds 202 can include one or more metallic particles 204, a polymeric layer 206, and a number of initiator molecules 208. In at least some examples, the framework compounds 202 can be produced according to the process 100 described with respect to FIG. 1 and correspond to the framework compounds 122.

In one or more examples, the process 200 can include, at 210, adding nucleotides to the initiator molecules 208 of the framework compounds 202. In various examples, nucleotides can be added to the framework compounds 202 in accordance with predetermined nucleic acid sequences 212. The predetermined nucleic acid sequences 212 can include nucleic acid sequences that are determined according to one or more encoding schemes in relation to digital data. The one or more encoding schemes can indicate one or more nucleotides that correspond to one or more digital data representations. For example, the one or more encoding schemes can indicate one or more nucleic acids that correspond to one or more combinations of 1s and 0s included in at least one of bits or bytes representing digital data. In this way, the order of adding nucleotides to the initiator molecules 208 is based on the predetermined nucleic acid sequences 212.

In one or more illustrative examples, the addition of nucleotides to the initiator molecules 208 according to the predetermined nucleic acid sequences 212 can include a stepwise process that includes a number of reaction cycles of an oligonucleotide synthesis process. The number of reaction cycles of the oligonucleotide synthesis process can correspond to a length of the predetermined nucleic acid sequences 212. In various examples, the length of the predetermined nucleic acid sequences 212 can correspond to the number of nucleotides in a chain of nucleotides. In one or more additional examples, the number of reaction cycles of the nucleotide addition process can correspond to a desired length of the synthesized oligonucleotides. Individual reaction cycles of the oligonucleotide synthesis process can include adding one or more reaction solutions that include a number of nucleotide building blocks and one or more enzymes to one or more polar solutions comprising the framework compounds 202. The composition of the one or more reaction solutions can facilitate the addition of the nucleotide building blocks to a 3′—OH end of the initiator molecules 208. In one or more illustrative examples, the composition of the one or more reaction solutions can lower the pKa of 3′—OH groups at the ends of the initiator molecules 208 in preparation for the covalent joining of the 3′—OH end of the initiator molecules 208 with the 5′ phosphate moieties of dNTPs included in the one or more synthesis solutions. The joining of the 3′—OH end of the initiator molecules 208 with the 5′ phosphate moieties of dNTPs can be facilitated by the one or more enzymes included in the one or more reaction solutions.

Each reaction cycle of adding one or more instances of a nucleotide to the framework compounds 202 can take place under a set of reaction conditions to facilitate the joining of one or more instances of a nucleotide to an intermediate oligonucleotide bound to the framework compounds 202. The reaction conditions can include a duration for individual reaction cycles and one or more reaction temperatures. In one or more examples, the duration of an individual reaction cycle to add one or more instances of a nucleotide to intermediate oligonucleotides bound to the framework compounds 202 can be from about 30 seconds to about 10 minutes, from about 1 minute to about 8 minutes, from about 2 minutes to about 6 minutes, from about 1 minute to about 3 minutes, from about 2 minutes to about 4 minutes, or from about 3 minutes to about 5 minutes.

In one or more additional examples, reaction temperatures for an individual reaction cycle of the nucleotide addition process can be from about 20° C. to about 45° C., from about 25° C. to about 40° C., from about 20° C. to about 30° C., from about 30° C. to about 40° C., or from about 35° C. to about 45° C. In various examples, the additional of one or more instances of a nucleotide to an intermediate oligonucleotide can be performed at atmospheric pressure.

Individual reaction cycles to add one or more instances of a nucleotide to intermediate oligonucleotides can be terminated by at least one of applying heat to the reaction mixture or adding a chelating agent. For example, individual reaction cycles of the nucleotide addition process can be terminated by heating the reaction mixture to temperatures from about 65° C. to about 100° C. for a duration from about 2 minutes to about 15 minutes. Additionally, individual reaction cycles of the nucleotide additional process can be terminated by adding ethylenediaminetetraacetic acid (EDTA) to the reaction mixture. In various examples, a final concentration of EDTA in the reaction mixture can be from about 20 millimolar (mM) to about 50 mM. Further, one or more washing solutions can be applied to a reaction vessel after the termination of an individual reaction cycle and before the start of a next reaction cycle that adds one or more instances of another nucleotide to the intermediate oligonucleotides bound to the framework compounds 202. In one or more examples, the one or more washing solutions can include a buffer solution comprising at least one of one or more exonucleases or one or more phosphatases. In at least some examples, the one or more washing solutions can be heated for a period of time. In one or more illustrative examples, the one or more washing solutions can be heated at temperatures from about 30° C. to about 95° C. for a duration from about 5 minutes to about 40 minutes.

In one or more examples, a first reaction cycle can add one or more instances of a first nucleotide 214 to at least a portion of the initiator molecules 208 to produce a first intermediate oligonucleotide. In at least some examples, the first reaction cycle can produce a homopolymer comprised of the first nucleotide 214. For example, the first reaction cycle can produce first intermediate oligonucleotides having from 1 to 10 instances of the first nucleotide 214 on the 3′ end of the first intermediate oligonucleotide, from 2 to 8 instances of the first nucleotide 214 on the 3′ end of the first intermediate oligonucleotide, from 3 to 6 instances of the first nucleotide 214 on the 3′ end of the first intermediate oligonucleotide, or from 2 to 5 instances of the first nucleotide 214 on the 3′ end of the first intermediate oligonucleotide. In one or more illustrative examples, when a first nucleotide in a predetermined nucleic acid sequence 212 is adenine (A), the one or more instances of the first nucleotide 214 of the first intermediate oligonucleotide can include A, AA, AAA, and so forth up to a threshold number of nucleotides.

Additional reaction cycles can be implemented to add other nucleotides to the first intermediate oligonucleotide until at least one reaction cycle has been performed in relation to the individual nucleotides of a predetermined nucleic acid sequence 212. To illustrate, a second reaction cycle can be performed to add one or more instances of a second nucleotide 216 to the first intermediate oligonucleotide to produce a second intermediate oligonucleotide, a third reaction cycle can be performed to add one or more instances of a third nucleotide 218 to the second intermediate oligonucleotide to produce a third intermediate oligonucleotide, and a fourth reaction cycle can be performed to add one or more instances of a fourth oligonucleotide 220 to the third intermediate oligonucleotide to produce a fourth intermediate oligonucleotide. Additional reaction cycles can continue to be performed until completion of one or more predetermined nucleic acid sequences 212 is reached.

In scenarios where the predetermined nucleic acid sequence 212 includes a consecutive number of a single nucleotide, multiple reaction cycles can be performed for each instance of the nucleotide in the predetermined nucleic acid sequence 212. In one or more additional examples, when the predetermined nucleic acid sequence 212 includes a consecutive number of a single nucleotide, a single reaction cycle can be performed with a longer duration. Performing a reaction cycle with a longer duration enables more instances of the nucleotide to be added to intermediate oligonucleotides. In various examples, a decoding scheme can indicate that a threshold number of instances of a nucleotide in the oligonucleotides synthesized using the framework compounds 202 corresponds to a single nucleotide in the predetermined nucleic acid sequences 212. For example, a decoding scheme can indicate that from 2 to 5 instances of a nucleotide in the oligonucleotides synthesized using the framework compounds 202 corresponds to a single nucleotide in the predetermined nucleic acid sequences 212. In these implementations, multiple iterations of a reaction cycle can be performed to add a number of instances of the nucleotide to the intermediate oligonucleotides that correspond to at least a minimum number of nucleotides to satisfy the threshold for the multiple instances of the nucleotide in the predetermined nucleic acid sequence 212. In these situations, a number of reaction cycles can be performed to add nucleotides to the intermediate oligonucleotides bound to the framework compounds 202 that corresponds to the number of nucleotides in the predetermined nucleic acid sequences 212. In one or more other examples, a single iteration of a reaction cycle can be performed for a duration that enables at least the threshold number of instances of the nucleotide to be added to an intermediate oligonucleotide to correspond to the multiple instances of the nucleotide in the predetermined nucleic acid sequence 212. In these scenarios, a number of reaction cycles can be performed to add nucleotides to the intermediate oligonucleotides bound to the framework compounds 202 that is less than the number of the common nucleotide in the predetermined nucleic acid sequences 212.

In one or more illustrative examples, for a predetermined nucleic acid sequence 212 having a sequence beginning with AGCC, a first reaction cycle can be performed to add multiple adenines to the initiator molecules 208 to produce a first intermediate oligonucleotide and a second reaction cycle can be performed to add multiple guanines to the first intermediate oligonucleotide to produce a second intermediate oligonucleotide. Subsequently, either multiple iterations of a reaction cycle to add cytosines to the second intermediate oligonucleotide can be performed to produce a third intermediate oligonucleotide and a fourth intermediate oligonucleotide or a single interaction of a reaction cycle to add cytosines to the second oligonucleotide can be performed for a greater duration than a single reaction cycle to produce a third intermediate oligonucleotide.

After a number of iterations of reaction cycles are performed to add nucleotides to the initiator molecules 208 of the framework compounds 202, oligonucleotides 224 can be produced that are bound to the framework compounds 202. In one or more examples, an oligonucleotide-metal-polymer complex 226 can be formed after a number of reaction cycles have been performed to synthesize the oligonucleotides 224. In one or more illustrative examples, a first batch of framework compounds can be subjected to a first group of reaction cycles to produce first oligonucleotides that correspond to a first encoded data nucleic acid sequence and a second batch of framework compounds can be subjected to a second group of reaction cycles to produce second oligonucleotides that correspond to a second encoded data nucleic acid sequence. In this way, a number of batches of oligonucleotides can be produced using framework compounds, where the number of batches corresponds to different predetermined nucleic acid sequences 212. Individual batches corresponding to an individual predetermined nucleic acid sequence 212 can include from 1000 oligonucleotides to 10,000 oligonucleotides to 100,000 oligonucleotides or more.

After the oligonucleotides 224 have been synthesized, the process 200 can include, at 228, separating the oligonucleotides 224 from the metal-polymer hybrid particles. In one or more examples, the oligonucleotides 224 can be separated from the metal-polymer hybrid particles using one or more separation solutions 230. The one or more separation solutions 230 can comprise an aqueous solution that includes one or more surfactants. The one or more surfactants can cause the electrostatic interactions between the oligonucleotides 224 and the polymeric layer 206 to break down enabling the oligonucleotides 224 to become unbound from the metal-polymer hybrid particles. In one or more additional examples, the one or more separation solutions 230 can include sodium dodecyl sulfate (SDS).

In one or more illustrative examples, an amount of one or more surfactants present in the one or more separation solutions 230 can be at least about 0.02% by total volume of the one or more separation solutions 230, at least about 0.05% by total volume of the one or more separation solutions 230, at least about 0.10% by total volume of the one or more separation solutions 230, at least about 0.15% by total volume of the one or more separation solutions 230, at least about 0.20% by total volume of the one or more separation solutions 230, at least about 0.25% by total volume of the one or more separation solutions 230, at least about 0.30% by total volume of the one or more separation solutions 230, at least about 0.35% by total volume of the one or more separation solutions 230, or at least about 0.40% by total volume of the one or more separation solutions 230. In one or more additional illustrative examples, an amount of one or more surfactants present in the one or more separations solutions 230 can be no greater than about 1% by total volume of the one or more separation solutions 230, no greater than about 0.90% by total volume of the one or more separation solutions 230, no greater than about 0.80% by total volume of the one or more separation solutions 230, no greater than about 0.70% by total volume of the one or more separation solutions 230, no greater than about 0.60% by total volume of the one or more separation solutions 230, or no greater than about 0.50% by total volume of the one or more separation solutions 230. In one or more further illustrative examples, an amount of one or more surfactants present in the one or more separation solutions 230 can be from about 0.02% to about 1% by total volume of the one or more separation solutions 230, from about 0.05% to about 0.80% by total volume of the one or more separation solutions 230, from about 0.10% to about 0.50% by total volume of the one or more separation solutions 230, from about 0.05% to about 0.40% by total volume of the one or more separation solutions 230, from about 0.05% to about 0.30% by total volume of the one or more separation solutions 230, or from about 0.20% to about 0.60% by total volume of the one or more separation solutions 230.

The one or more separation solutions 230 can be applied to the oligonucleotide-metal-polymer complexes 226 at temperatures from about 15° C. to about 40° C., temperatures from about 20° C. to about 30° C., or temperatures from about 25° C. to about 35° C. for a duration from about 30 seconds to about 10 minutes, from about 1 minute to about 8 minutes, from about 2 minutes to about 6 minutes, or from about 1 minute to about 5 minutes to separate the oligonucleotides 224 from the metal-polymer hybrid particles. In various examples, the one or more separation solutions 230 can be applied to the oligonucleotide-metal-polymer complexes 226 in a same container as a reaction vessel in which the nucleotides were added to the framework compounds 202. In one or more additional examples, the one or more separation solutions 230 can be applied to the oligonucleotide-metal-polymer complexes 226 in a container that is different from a reaction vessel in which the nucleotides were added to the framework compounds 202.

At 232, the process 200 can include storing the oligonucleotides 224 after the oligonucleotides 224 have been separated from the metal-polymer hybrid particles. The oligonucleotides 224 can be stored in a storage container 234 that includes a storage solution 236. Oligonucleotides 224 can be stored in the storage container until a request is received to retrieve at least a portion of the digital data encoded by the predetermined nucleic acid sequences 212.

The oligonucleotides 224 produced using the process 200 can include one or more sections. For example, the oligonucleotides 224 can include an initiator sequence 238 that corresponds to a sequence of an initiator molecule 208 used to synthesize the oligonucleotides 224. Additionally, the oligonucleotides 224 can include an adapter sequence and/or an identifier sequence 240. The adapter/identifier sequence 240 can include a nucleotide sequence that can be used during one or more sequencing processes that are implemented to retrieve digital data stored by the oligonucleotides 224. For example, the adapter/identifier sequence 240 can correspond to sequences of one or more primers used in the one or more sequencing processes implemented to retrieve digital data stored by the oligonucleotides 224. The adapter/identifier sequence 240 can additionally, or alternatively, include information that indicates an order for a given oligonucleotide in a group of oligonucleotides that encode digital data. To illustrate, a string of digital data can be encoded by a number of oligonucleotides that are arranged in a given order. In these situations, the oligonucleotides used to encode the string of digital data are decoded according to the order followed during the encoding process. The adapter/identifier sequence 240 can correspond to the location within the given string of digital data indicated by the particular oligonucleotide 224 such that the digital data encoded by the particular oligonucleotide 224 is decoded according to the order followed during the encoding process. In various examples, the adapter/identifier sequence 240 can be included in the predetermined nucleic acid sequences 212. Further, the adapter/identifier sequence 240 can be located after the initiator sequence 238 in the string of nucleotides comprising the oligonucleotides 224.

The oligonucleotides 224 can also include a payload sequence 242. The payload sequence 242 can correspond to digital data encoded by the predetermined nucleic acid sequences 212. In various examples, the payload sequence 242 can be located after the adapter/identifier sequence 240. In one or more additional examples, one or more additional sequence portions can be located in the string of nucleotides comprising the oligonucleotides 224 after the payload sequence 242. For example, an adapter/identifier sequence 240 can be located before and after the payload sequence 242 within the string of nucleotides comprising the oligonucleotides 224.

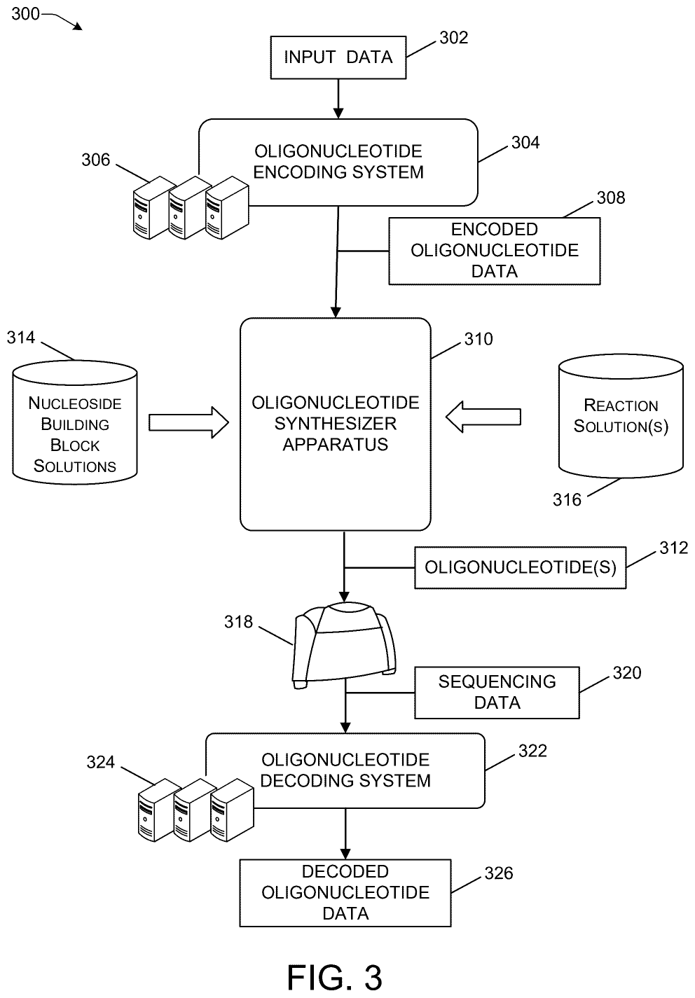

FIG. 3 is diagram of an architecture 300 to encode data using oligonucleotides synthesized using metal-polymer framework compounds, in accordance with one or more implementations. The architecture 300 can include input data 302. The input data 302 can include information that is to be encoded using oligonucleotides. In one or more examples, the input data 302 can include binary data stored in one or more data files. In one or more additional examples, the input data 302 can include ternary coded data. The input data 302 can correspond to digital information stored in one or more documents, one or more databases, one or more applications, one or more media files, or one or more combinations thereof.

The architecture 300 can include an oligonucleotide encoding system 304 that obtains the input data 302 The oligonucleotide encoding system 304 can be implemented by one or more computing devices 306. For example, the one or more computing devices 306 can include at least one of one or more desktop computing devices, one or more mobile computing devices, or one or more server computing device. In various examples, at least a portion of the one or more computing devices 306 can be included in a remote computing environment, such as a cloud computing environment. The oligonucleotide encoding system 304 can analyze the input data 302 and generate oligonucleotide sequences that encode the data. To illustrate, the oligonucleotide encoding system 304 can generate encoded oligonucleotide data 308 that corresponds to the input data 302. The oligonucleotide encoding system 304 can analyze the input data 302 using one or more encoding algorithms to generate the encoded oligonucleotide data 308.

The oligonucleotide sequences included in the encoded oligonucleotide data 308 can correspond to DNA sequences, RNA sequences, or combinations of DNA sequences and RNA sequences. In one or more examples, one or more portions of oligonucleotide sequences included in the encoded oligonucleotide data 308 that correspond to DNA can include sequences represented by the four bases found naturally occurring in DNA: cytosine (C), guanine (G), adenine (A), and thymine (T). One or more portions of oligonucleotide sequences included in the encoded oligonucleotide data 308 that correspond to RNA can include sequences represented by the four bases found naturally occurring in RNA: cytosine (C), guanine (G), adenine (A), and uracil (U). In at least some examples, the oligonucleotide sequences included in the encoded oligonucleotide data 308 can include single stranded oligonucleotide sequences. In one or more additional examples, the oligonucleotide sequences included in the encoded oligonucleotide data 308 can include double stranded sequences. In one or more further examples, the oligonucleotide sequences included in the encoded oligonucleotide data 308 can include a combination of single stranded sequences and double stranded sequences.

The architecture 300 can include an oligonucleotide synthesizer apparatus 310 that synthesizes oligonucleotides 312 based on the encoded oligonucleotide data 308. The oligonucleotide synthesizer apparatus 310 can include one or more containers to produce oligonucleotides. In various examples, the oligonucleotide synthesizer apparatus 310 can include one or more reaction vessels in which one or more reactions take place in the production of the oligonucleotides 312. In one or more illustrative examples, the oligonucleotide synthesizer apparatus 310 can include a number of wells and/or a number of channels in which one or more reactions can take place to produce the oligonucleotides 312.

The oligonucleotide synthesizer apparatus 310 can implement enzymatic synthesis of oligonucleotides. The enzymatic synthesis of the oligonucleotides 312 by the oligonucleotide synthesizer apparatus 310 can add deoxynucleoside triphosphate (dNTP) building blocks in a 5′ to 3′ direction to intermediate oligonucleotide chains. In various examples, the enzymatic synthesis of the oligonucleotides 312 by the oligonucleotide synthesizer apparatus 310 can add dNTP building blocks in a 5′ to 3′ direction to intermediate oligonucleotide chains. In one or more examples, deoxynucleoside triphosphate building blocks can be added to intermediate oligonucleotide chains in an order that corresponds to the oligonucleotide sequences included in the encoded oligonucleotide data 308. The deoxynucleoside triphosphate used to synthesize the oligonucleotides 312 can be included in nucleoside building block solutions 314. In one or more examples, the deoxynucleoside triphosphate building blocks can comprise deoxynucleoside triphosphates that have been unaltered from their natural state. In one or more additional examples, the deoxynucleoside triphosphate building blocks can include deoxynucleoside triphosphates that have been altered from their natural state. For example, modifications can be made to one or more functional groups of natural deoxynucleoside triphosphates that do not hinder interactions with enzymes used in the oligonucleotide synthesis process to produce modified deoxynucleoside triphosphates. In one or more additional examples, modifications can be made to one or more functional groups of natural deoxynucleoside triphosphates to control the addition of nucleotides to growing oligonucleotide chains. To illustrate, modified deoxynucleoside triphosphates can be used to produce the oligonucleotides 312 by adding single nucleotides to a growing oligonucleotide chain.

In various examples, the nucleoside building block solutions 314 can include multiple solutions with individual solutions including a single deoxynucleoside triphosphate. For example, the nucleoside building block solutions 314 can include a first solution that includes an amount of deoxyadenosine triphosphate, a second solution that includes an amount of deoxythymidine triphosphate, a third solution that includes an amount of deoxyguanosine triphosphate, and a fourth solution that includes an amount of deoxycytidine triphosphate. In situations where the oligonucleotides 312 include RNA sequences, the nucleoside building block solutions 314 can include deoxyuridine triphosphate. The nucleoside building block solutions 314 can be solutions that also include at least one of a buffer or a salt. In at least some examples, the nucleoside building block solutions 314 can comprise aqueous solutions.

The architecture 300 can also include reaction solutions 316. The reaction solutions 316 can cause one or more chemical reactions to take place within the oligonucleotide synthesizer apparatus 310 to produce the oligonucleotides 312. For example, the reaction solutions 316 can include one or more enzyme solutions. The one or more enzyme solutions can include one or more enzymes that can facilitate the additional of nucleotides to a chain of nucleotides. In one or more examples, the one or more enzymes can include at least one of a polynucleotide phosphorylase (PNPase), a T4 RNA ligase (T4Rnl), or a terminal deoxynucleotidyl transferase (TdT). In various examples, the one or more enzyme solutions can include cations to aid in the enzymatic production of oligonucleotides. To illustrate, the one or more enzyme solutions can include at least one of Co²⁺, Mg²⁺, Mn²⁺, or Zn²⁺.

Additionally, the reaction solutions 316 can include one or more solutions that include framework compounds that can be used to synthesize the oligonucleotides 312. The framework compounds can include metallic particles that have a polymeric coating. Initiator molecules can be disposed on the polymeric coating that comprise starting sequences that can be used by one or more enzymes to add nucleotides according to a given nucleic acid sequence. In one or more illustrative examples, the framework compounds can correspond to the framework compounds 122 and 202 described with respect to FIG. 1 and FIG. 2. In various examples, the framework compounds can be disposed in an aqueous solution that comprises one or more polar solvents.

In one or more examples, at least one of the nucleoside building block solutions 314 or the reaction solutions 316 can include one or more buffers. The one or more buffers can include at least one of a Tris(Hydroxymethyl)aminomethane buffer, a (4-(2-hydroxyethyl)-1-piperazineethanesulfonic acid) (HEPES) buffer, a N-cyclohexyl-3-aminopropanesulfonic acid (CAPS) buffer, a 4-(2-hydroxyethyl)-1-piperazine propanesulfonic acid (EPPS) buffer, a 2-(N-morpholino)ethanesulfonic acid (MES) buffer, a [3-(N-morpholino)propanesulfonic acid] (MOPS) buffer, a (piperazine-N,N′-bis(2-ethanesulfonic acid)) (PIPES) buffer, tris(hydroxymethyl)methylamino]propanesulfonic acid (TAPS) buffer, or a 2-[Tris(hydroxymethyl)-methylamino]-ethanesulfonic acid (TES) buffer. In one or more additional examples, at least one of the nucleoside building block solutions 314 or the reaction solutions 316 can include other components, such as one or more salts, one or more surfactants, one or more alcohols, one or more stabilizing agents, and/or one or more reducing agents. To illustrate, at least one of the nucleoside building block solutions 314 or the reaction solutions 316 can include at least one of (tris(2-carboxyethyl)phosphine) (TCEP), dimethyl sulfoxide (DMSO), Ethylenediaminetetraacetic acid (EDTA), ethyl acetate, acetic acid, isopropanol, ethanol, methanol, glycerol, potassium salts, sodium salts, dithiothreitol (DTT), or P-mercaptoethanol.

Each cycle of the oligonucleotide synthesis process can cause one or more instances of a nucleotide to be added to intermediate oligonucleotide chains. The order of the nucleotides added to the intermediate oligonucleotide chains is based on the oligonucleotide sequences included in the encoded oligonucleotide data 308. In one or more illustrative examples, when a next nucleotide to be added to one or more intermediate oligonucleotide chains is adenine, a cycle of the oligonucleotide synthesis process can be performed with a deoxyadenosine triphosphate solution and when a next nucleotide to be added to one or more intermediate oligonucleotide chains is thymine, a cycle of the oligonucleotide synthesis process can be performed with a deoxythymidine triphosphate solution. Additionally, when a next nucleotide to be added to one or more intermediate oligonucleotide chains is guanine, a cycle of the oligonucleotide synthesis process can be performed with a deoxyguanosine triphosphate solution and when a next nucleotide to be added to one or more intermediate oligonucleotide chains is cytosine, a cycle of the oligonucleotide synthesis process can be performed with a deoxycytidine triphosphate solution.

After synthesis of oligonucleotide chains in the oligonucleotide synthesizer apparatus 310 using the framework compounds is complete, the completed oligonucleotide chains can be separated from the framework compounds to produce the oligonucleotides 312. In various examples, the completed oligonucleotide chains can be separated from the framework compounds using one or more solutions comprising one or more surfactants. In one or more examples, the oligonucleotides 312 can be stored under conditions that minimize degradation of the oligonucleotides 312. For example, the oligonucleotides 312 can be stored at temperatures from about −10° C. to −80° C. or from −20° C. to −70° C. in a slightly basic solution. To illustrate, the oligonucleotides 312 can be stored in a solution having a pH from about 7.8 to about 8.2 that includes at least one of Tris (hydroxymethyl) aminomethane hydrochloride or Ethylenediaminetetraacetic acid (EDTA). In one or more additional examples, the oligonucleotides 312 can undergo one or more drying processes and be stored at temperatures from about 10° C. to about 25° C.

The storage of the oligonucleotides 312 in a suitable environment enables the data encoded by the oligonucleotides to be stored until a request is received to retrieve the encoded data. To retrieve data encoded by at least a portion of the oligonucleotides 312, the portion of the oligonucleotides 312 that corresponds to the data being retrieved are provided to a sequencing apparatus 318. The sequencing apparatus 318 can perform one or more sequencing operations to generate sequencing data 320. The sequencing data 320 can include sequencing reads that correspond to the nucleotide sequences of at least a portion of the oligonucleotides 312. The sequencing apparatus 318 can implement one or more next generation sequencing techniques. Next generation sequencing techniques can include post-Sanger, high throughput sequencing techniques that sequence millions of nucleotide fragments in parallel. In various examples, the sequencing apparatus 318 can implement other sequencing techniques, such as Sanger sequencing, nanopore sequencing, or single molecular real-time sequencing. In one or more illustrative examples, the sequencing operations can be performed according to techniques described in “High-Throughput Sequencing Technologies” by Jason A. Reuter et al., Mol. Cell. 2015 May 21; 58)4); 586-597.

The sequencing data 320 can be analyzed by an oligonucleotide decoding system 322 that is implemented by one or more computing devices 324. The oligonucleotide decoding system 322 can implement one or more computational algorithms to generate decoded oligonucleotide data 326 from the sequencing data 320. For example, the oligonucleotide decoding system 322 can analyze sequencing reads to determine at least one of the bits or bytes encoded by the respective sequence reads to produce the decoded oligonucleotide data 326. In one or more examples, the decoded oligonucleotide data 326 can be assembled into a data file that can be read by a computing device. In one or more illustrative examples, the decoded oligonucleotide data 326 can be used to generate a portion of a database that corresponds to at least a portion of the input data 302.

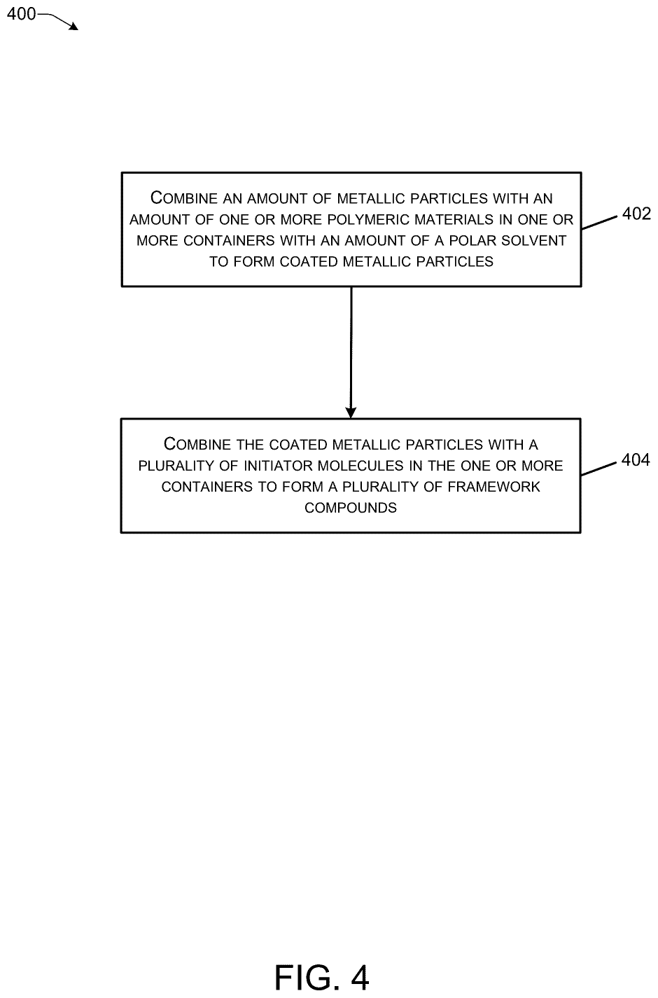

FIG. 4 is a flow diagram of an example method 400 to make metal-polymer framework compounds for the production of oligonucleotides, in accordance with one or more implementations. The method 400 can include, at 402, combining an amount of metallic particles with an amount of one or more polymeric materials in one or more containers with an amount of one or more polar solvents to form coated metallic particles. Individual coated metallic particles can comprise a polymeric layer of the one or more polymeric materials disposed on a metallic particle and the polymeric layer can be bound to the metallic particle by first electrostatic interactions. In one or more examples, the one or more polar solvents can include one or more aqueous solutions. In one or more additional examples, the metallic particles can include negatively charged metallic particles in the polar solvent and the one or more polymeric materials can comprise one or more positively charged functional groups in the polar solvent. In these situations, the first electrostatic interactions can comprise the negatively charged metallic particles interacting with a first portion of the one or more positively charged functional groups of the one or more polymeric materials.

In one or more examples, the metallic particles can have magnetic properties. To illustrate, the metallic particles can include superparamagnetic metallic particles. In one or more additional examples, the amount of metallic particles can comprise a metal oxide. For example, the amount of metallic particles can comprise an iron oxide. In one or more illustrative examples, the amount of metallic particles can include at least one of Fe₂O₃ or Fe₃O₄. In at least some examples, the metallic particles can have a spherical shape. In various examples, the metallic particles can have diameters from about 80 nanometers to about 150 nanometers.

In one or more examples, the polymeric layer can have a thickness from about 10 nanometers to about 50 nanometers. Additionally, the one or more polymeric materials can have a number average molecular weight from about 5 kilodaltons (kDa) to about 15 kDa. In one or more additional illustrative examples, the one or more polymeric materials can include at least one of Poly(N,N-dimethylaminoethyl methacrylate, polyethylenimine, poly-L-lysine, polyvinylamine, or polyallylamine. In various examples, a ratio of a weight of the amount of the one or more polymeric materials to a weight of the amount of the metallic particles can be from about 3:1 to about 12:1.

Additionally, the method 400 can include, at 404, combining the coated metallic particles with a plurality of initiator molecules in the one or more containers to form a plurality of framework compounds. In at least some examples, at least a portion of the plurality of framework compounds can have a spherical shape and have diameters from about 100 nanometers to about 200 nanometers. In one or more examples, the framework compounds can be included in a formulation that comprises one or more polar solvents. In one or more illustrative examples, the one or more polar solvents can include H₂O. In various examples, a concentration of the plurality of framework compounds in the one or more polar solvents can be from about 10 milligrams (mg) to 30 mg of the plurality of framework compounds to 1 milliliter (mL) of the one or more polar solvents.

In various examples, a number of nucleotides can be added to individual initiator molecules of the plurality of initiator molecules to produce a plurality of oligonucleotides bound to the polymeric layer of the framework compounds. A separation solution including an amount of a surfactant can be added to the container to separate the oligonucleotides from the polymeric layer. In one or more illustrative examples, the separation solution can comprise at least about 0.03% by volume of sodium dodecyl sulfate.

The plurality of initiator molecules can comprise a plurality of nucleotides and the plurality of initiator molecules can be bound to the coated metallic particles by second electrostatic interactions. In one or more examples, the plurality of initiator molecules can comprise one or more negatively charged functional groups in the polar solvent. Additionally, the second electrostatic interactions can comprise the one or more negatively charged functional groups of the plurality of initiator molecules interacting with a second portion of the one or more positively charged functional groups of the one or more polymeric materials.

In at least some examples, the plurality of initiator molecules can include from 2 nucleotides to 30 nucleotides. Additionally, the plurality of nucleotides of the plurality of initiator molecules can include at least a portion of a nucleotide sequence of an M13 phage genome. In one or more examples, a ratio of a number of moles of the plurality of initiator molecules relative to a number of moles of the one or more polymeric materials in the polar solvent is from about 3:1 to about 6:1. Further, a ratio of a weight of the one or more polymeric materials of the polymeric layer relative to a weight of the initiator molecules can be from about 10:1 to about 18:1.

In one or more examples, one or more mixing devices can be applied to combine the amount of metallic particles with the amount of one or more polymeric materials. Additionally, the one or more mixing devices can be applied to combine the coated metallic particles with the plurality of initiator molecules. In one or more illustrative examples, the one or more mixing devices can include at least one of a mechanical stir bar, a paddle, or a sonicator. In various examples, the one or more mixing devices are applied to combine the amount of metallic particles with the amount of one or more polymeric materials for a first period of time from about 30 seconds to about 5 minutes. Further, the one or more mixing devices can be applied to combine the amount of metallic particles with the amount of one or more polymeric materials for a second period of time from about 30 seconds to about 5 minutes. In at least some examples, the coated metallic particles and the plurality of framework compounds can be formed in the amount of the one or more polar solvents at temperatures from about 15° C. to about 30° C.

In various examples, the oligonucleotides can correspond to nucleotide sequences that encode an amount of digital data. In this way, the number of nucleotides added to the individual initiator molecules of the plurality of initiator molecules to produce the plurality of oligonucleotides correspond to at least a portion of the nucleotide sequences that encode the digital data. In one or more examples, one or more sequencing operations can be performed with respect to at least a portion of the plurality of oligonucleotides to determine nucleotide sequences of the at least a portion of the plurality of oligonucleotides. The nucleotide sequences can then be analyzed according to a decoding scheme to determine one or more portions of the digital data that correspond to the at least a portion of the plurality of oligonucleotides.

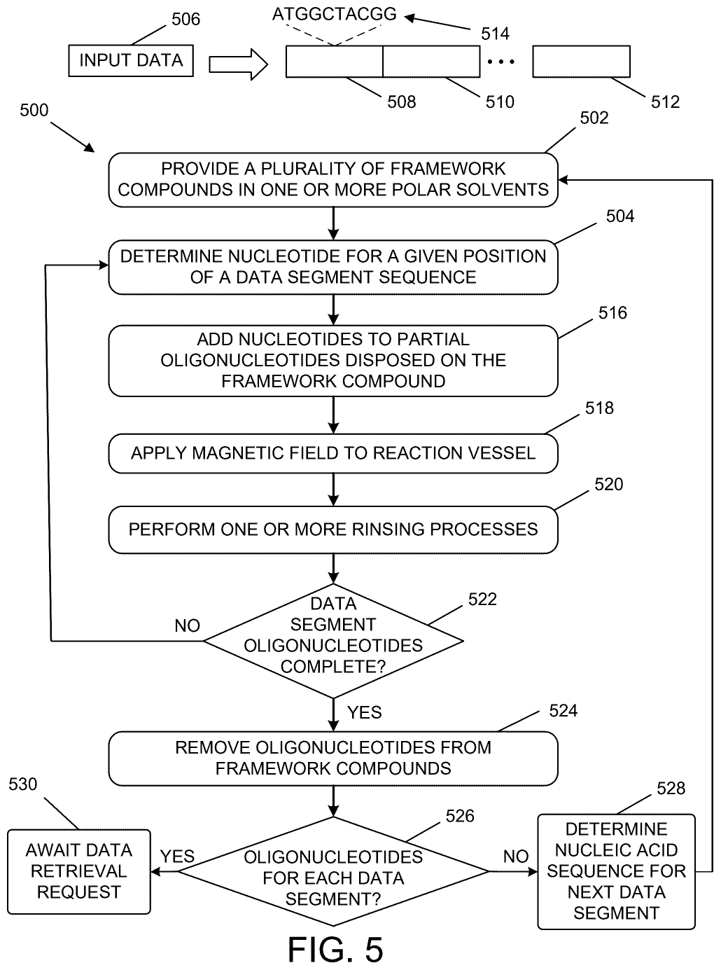

FIG. 5 is a flow diagram of an example method 500 to synthesize oligonucleotides using metal-polymer framework compounds, in accordance with one or more implementations. The method 500 can include, at 502, providing a plurality of framework compounds in one or more polar solvents. The one or more polar solvents comprising the plurality of framework compounds can be disposed in one or more containers. The one or more containers can be a part of an oligonucleotide synthesis apparatus. In one or more examples, the plurality of framework compounds can include a number of coated metallic particles having a plurality of initiator molecules bound to the number of coated metallic particles. Individual coated metallic particles can comprise a metallic particle coated with a polymeric layer that includes one or more polymeric materials. In one or more illustrative examples, the plurality of framework compounds can include framework compounds produced according to the methods described in relation to FIG. 1 and FIG. 4.

The method 500 can also include, at 504, determining a nucleotide for a given position of a data segment sequence to determine one or more nucleotide sequences that correspond to oligonucleotides to be synthesized using the framework compounds. In one or more examples, the data segment sequence can correspond to a portion of input data 506. The input data 506 can include digital data stored in one or more data files. The input data 506 can correspond to digital information stored in one or more documents, one or more databases, one or more applications, one or more media files, or one or more combinations thereof. The input data 506 can be represented by a number of nucleotide sequences. For example, discrete portions of the input data 506 can be represented by individual nucleotide sequences. To illustrate, the input data 506 can include a first data segment 508, a second data segment 510, up to an Nth data segment 512. In the illustrative example of FIG. 5, the first data segment 508 is represented by a nucleotide sequence representation 514. The second data segment 510 up to the Nth data segment 512 can also be represented by individual nucleotide sequences that encode the input data 506.

In one or more examples, the input data 506 can include digital data that has been generated by one or more applications executed by one or more computing devices. In various examples, the input data 506 can be represented according to one or more positional number systems. In at least some examples, the input data 506 can include a string of alphanumeric characters that represent the digital data. In one or more illustrative examples, a binary number system can be used to represent the input data 506. In these scenarios, the input data 506 can include a number of bits and a number of bytes. In one or more additional illustrative examples, the input data 506 can be represented by a hexadecimal number system. In one or more further illustrative examples, the input data 506 can be represented by an octal number system. In still other illustrative examples, the input data 506 can be represented by a decimal number system.

An encoding process can be used to generate oligonucleotide sequences, such as the nucleotide sequence representation 514, based on the input data 506. The encoding process can generate an encoded nucleotide sequence representation from the string of characters corresponding to the input data 506. In one or more examples, a first encoding process can transform a first string of characters corresponding to the first data segment 508 to an additional string of characters comprising the nucleotide sequence representation 514 with individual characters of the nucleotide sequence representation 514 being represented by nucleotides included in at least one of DNA or RNA. In this way, a first encoding process can generate the encoded nucleotide sequence representation 514 to include a string of characters that includes one or more A's, one or more G's, one or more C's, one or more T's, and, in cases where the encoded nucleotide sequence representation corresponds to RNA, one or more U's instead of one or more T's. The encoding process can include transforming combinations of characters included in a first string of characters representing the first data segment 508 to one or more characters included in DNA and/or RNA sequences to generate the encoded nucleotide sequence representation 514 according to an encoding scheme. In one or more illustrative examples, the encoding process to transform a 00 combination in a first string of characters corresponding to the first data segment 508 as an A in the additional string of characters comprising the nucleotide sequence representation 514, a 01 combination in the first string of characters corresponding to the first data segment 508 as a T in the additional string comprising the nucleotide sequence representation 514, a 10 combination in a first string of characters corresponding to the first data segment 508 as a G in the additional string of characters comprising the nucleotide sequence representation 514, and a 11 combination in a first string of characters corresponding to the first data segment 508 as a C in the additional string of characters comprising the nucleotide sequence representation 514. Although an example encoding scheme has been described above as an illustrative example, a number of different encoding schema can be implemented by one or more encoding processes to generate the encoded nucleotide sequence representation 514 from the portion of the input data 506 corresponding to the first data segment 508.

At 516, the method 500 can include adding a nucleotide to partial oligonucleotides disposed on the framework compound. For example, in a first step of an oligonucleotide synthesis process, one or more nucleotides can be added to the initiator molecules bound to the polymeric material of the framework compounds, where the one or more nucleotides correspond to a first character of the nucleotide sequence representation 514 encoding the first segment 508. In the illustrative example of FIG. 5, a first step of an oligonucleotide synthesis process can include adding one or more adenosines (A's) to initiator molecules disposed on the polymeric coating of the framework compounds. In various examples, a number of nucleotides added to the initiator molecules can be from 1 to 10 instances of the nucleotide. In this way, a homopolymer can be produced at 516 to add nucleotides to the initiator molecules of the framework compounds.

In various examples, the nucleotides can be added by combining a deoxynucleoside triphosphate (dNTP) solution to one or more containers in which the plurality of framework compounds is present. Additionally, the nucleotides are added to the individual initiator molecules by providing one or more enzymes to the one or more containers in conjunction with the plurality of dNTP solutions. In one or more illustrative examples, the one or more enzymes can include terminal deoxynucleotidyl transferase (TdT).

In various examples, the dNTP solution can be one of a number of dNTP solutions that can be added to the one or more containers to add nucleotides depending on the nucleotide to be added according to the nucleotide sequence representation 514. For example, a first dNTP solution comprising an amount of deoxyadenosine triphosphate can be added to the one or more containers when an adenosine is located at a current position of the nucleotide sequence representation 514. Additionally, the number of dNTP solutions that can be added to the one or more containers when thymine is located at a current position of the nucleotide sequence representation 514 can include a second dNTP solution comprising an amount of deoxythymidine triphosphate. Further a third dNTP solution comprising an amount of deoxyguanosine triphosphate can be added to the one or more containers when a guanine is located at a current position of the nucleotide sequence representation 514 and a fourth dNTP solution comprising an amount of deoxycytidine triphosphate can be added to the one or more containers when cytosine is located at a current position of the nucleotide sequence representation 514. A first intermediate framework compound that is bound to the polymeric layer of the framework compounds can be produced at 516, where the first intermediate framework compound can include the initiator molecules and a number of the first nucleotide in the nucleotide sequence representation 514 added to the initiator molecules.

The method 500 can also include, at 516, applying a magnetic field to a reaction vessel. In one or more examples, the magnetic field can have a value that modifies the magnetic pole of the metallic particles included in the framework compounds. In this way, the first intermediate compounds can be moved by the magnetic field. In one or more additionally examples, the first intermediate framework compounds can be moved toward a wall of the reaction vessel in the presence of the magnetic field. In one or more further examples, the first intermediate framework compounds can be bound to one or more walls of the reaction vessel in the presence of the magnetic field.

Further, at 518, the method 500 can include performing one or more rinsing processes. The one or more rinsing processes can include providing one or more rinse solutions to the reaction vessel. In one or more examples, the one or more rinsing solutions can be provided to the reaction vessel while the magnetic field is being applied to the reaction vessel. In this way, providing the one or more rinsing solutions to the reaction vessel can cause one or more enzyme solutions and/or one or more dNTP solutions to be washed out of the reaction vessel while the first intermediate framework compounds are bound to one or more walls of the reaction vessel. At least portions of a formulation comprising the first intermediate framework compounds can also be removed from the reaction vessel in response to the one or more rinsing solutions being provided to the reaction vessel and while the magnetic field is being applied to the reaction vessel. In at least some examples, an amount of the one or more polar solutions in which the first intermediate framework compounds were disposed can be added to the reaction vessel after the initial reaction solutions have been removed from the reaction vessel. In this way, the reaction vessel and the first intermediate framework compounds can be ready for a next operation in the method 500 to produce oligonucleotides using an enzymatic process.

At 522, the method 500 can include determining whether or not oligonucleotides have been produced that correspond to each position of the nucleotide sequence corresponding to a given data segment. In situations where the oligonucleotides are not complete, the method 500 can return to 504 where a nucleotide for a next position of a data segment sequence is determined and the operations 516, 518, and 520 are repeated to add homopolymers of another nucleotide to the intermediate oligonucleotides bound to the polymeric layer of the framework compounds. Continuing with the illustrative example from above in relation to the nucleotide sequence representation 514, after forming the first intermediate framework compounds that include the initiator molecules and a number of adenosine homopolymers, the operations 516, 518, and 520 can be performed to add homopolymers of thymine to the first intermediate framework compounds. In these scenarios, a second dNTP solution can be added to the reaction vessel that includes a second deoxynucleoside triphosphate that corresponds to the next nucleotide, in this case thymine, to add one or more instances of thymine to the growing oligonucleotide chains to produce second intermediate framework compounds. In various examples, a second amount of the one or more enzymes can be added concurrently to the reaction vessel with an amount of a deoxythymidine triphosphate.

In situations where the oligonucleotides for a given data segment of the input data 506 have been completed, the method 500 can move to 524. At 524, the method 500 can include removing the completed oligonucleotides from the framework compounds. In one or more examples, the oligonucleotides can be removed from the framework compounds using one or more separation solutions. In various examples, the one or more separation solutions can include one or more surfactants. In one or more illustrative examples, the one or more separation solutions can include sodium dodecyl sulfate. In one or more additional examples, after removal of the oligonucleotides from the framework compounds, the oligonucleotides can be stored in one or more storage containers.

The method 500 can then proceed to 526 where a decision can be made to determine whether oligonucleotides for each data segment of the input data 506 have been produced using framework compounds. In instances where oligonucleotides have not been produced for each data segment corresponding to the input data 506, the method 500 can move to 528. At 528, the method 500 can include determining a nucleic acid sequence for a next data segment that corresponds to the input data 506. For example, after oligonucleotides have been produced that correspond to an encoding of the first data segment 508, a nucleotide sequence corresponding to the second data segment 510 can be determined. The method 500 can then return to 502 to produce oligonucleotides that correspond to a nucleotide sequence related to the second data segment 510. In various examples, the oligonucleotides that correspond to a nucleotide sequence related to the second data segment 510 can be produced by implementing the method 500 with respect to a new batch of framework compounds.

In scenarios where oligonucleotides have been produced for each data segment related to the input data 506, the method 500 can move to 530. At 530, the stored oligonucleotides can await one or more data retrieval requests. A data retrieval request can correspond to retrieving an amount of data included in the input data. In response to a data retrieval request, an amount of a solution that includes the completed oligonucleotides produced by the method 500, can be obtained and subjected to one or more sequencing processes. The sequencing reads generated by the one or more sequencing processes can then be analyzed in relation to one or more decoding schemes. The one or more decoding schemes can be used to determine a string of characters in relation to the input data, such as a string of binary characters, that correspond to the nucleotide sequences of the sequencing reads. After producing the string of binary characters, the decoded output data can be provided to one or more computing devices. In one or more examples, the decoded output data can be used to determine data displayed in one or more user interfaces. In one or more additional examples, the decoded output data can be accessible to one or more computing device applications.

### EXAMPLES

Various aspects of the present invention can be better understood by reference to the following Examples which are offered by way of illustration. The inventive concepts described in the application are not limited to the Examples given herein.

### Example 1

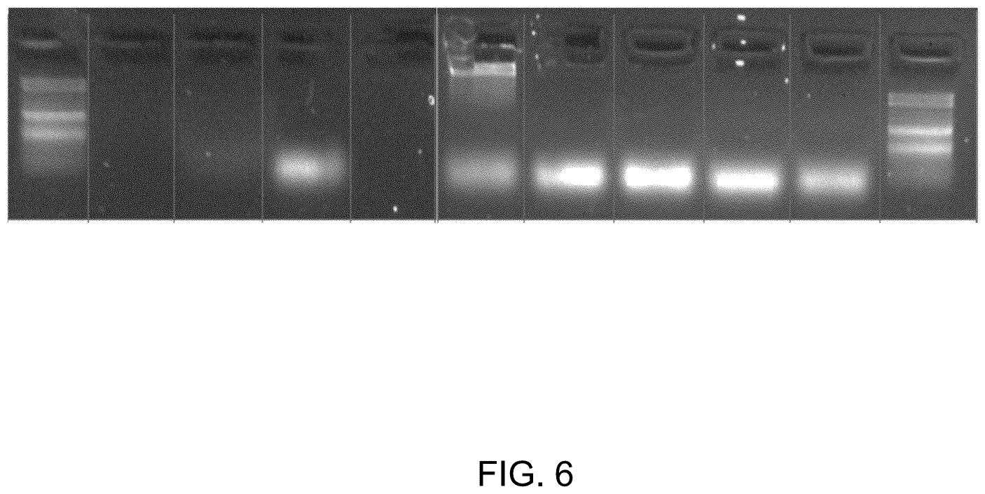

FIG. 6 includes an image of an agarose gel after performing an electrophoresis gel representing a number of combinations of materials used to produce framework compounds according to implementations described herein. Gel electrophoresis was performed to evaluate the functionalization of nanoparticles and oligonucleotides. The framework compounds were prepared and loaded onto 3% agarose gels with 50% glycerol (2 μL). The analysis occurred in 1×Tris-acetate-EDTA (TAE) buffer at 100 V for 40 minutes in a 15 cm electrophoresis chamber length.

Lane 1 of the agarose gel includes a control solution. The control solution is related to verifying if the run occurred as expected and if the size of the initiator is the expected size. Lane 2 of the agarose gel includes a first concentration of sodium dodecyl sulfate and Lane 3 of the agarose gel includes a second concentration of sodium dodecyl sulfate that is greater than the first concentration. In particular, Lane 2 includes a minimum concentration of sodium dodecyl sulfate and Lane 3 includes a maximum concentration of sodium dodecyl sulfate. Lane 4 of the agarose gel includes a solution that includes an amount of M13 initiator molecules. Lane 5 of the agarose gel includes an amount of coated metallic particles formed according to implementations described herein as described herein. The coated metallic particles included iron oxide nanoparticles coated with a layer of PDMEAMA. Lanes 6-10 of the agarose gel include increasing amounts of an aqueous sodium dodecyl sulfate (SDS) with the same amounts of initiator molecules and coated metallic particles. The aqueous SDS solution includes about 0.4% by volume SDS. Lane 11 of the agarose gel also includes an amount of a control solution.

The agarose gel shown in FIG. 6 is meant to show that increasing amounts of initiator molecules are released from Lanes 6-10 as the amounts of SDS present in the solution also increase. That is, the signal for the initiator molecules grows stronger as the amount of SDS solution present in a given lane of the agarose gel increases. These results indicate that the initiator molecules are bound to the coated metallic particles by electrostatic interactions that are able to be broken down with relatively mild surfactants, such as the aqueous solution including SDS used in Lanes 6-10.

### Example 2

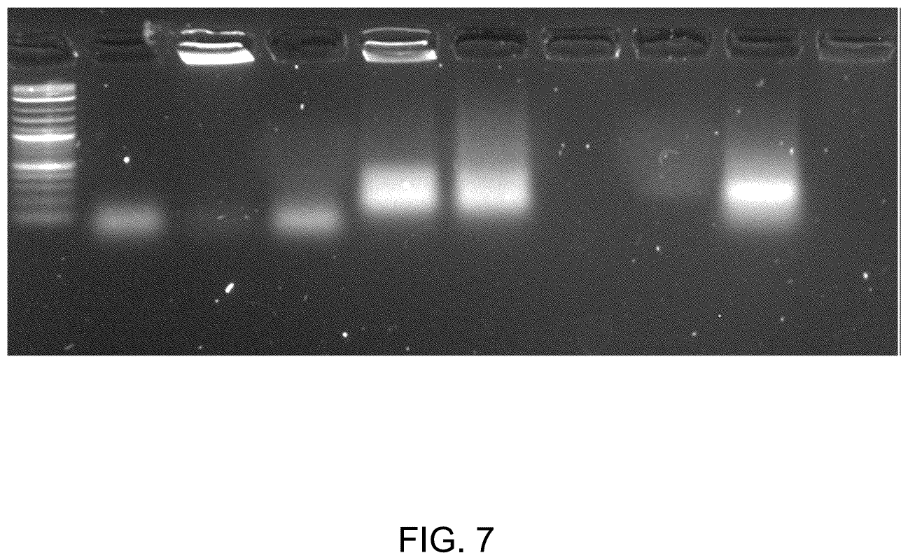

FIG. 7 includes an image of an agarose gel after performing an electrophoresis gel representing a number of combinations of materials used to synthesize oligonucleotides with framework compounds according to implementations described herein. Lane 1 of the agarose gel includes a control solution and Lane 2 includes a solution with an amount of M13 initiator molecules. The control solution in Lane 1 is related to verifying if the run occurred as expected and if the size of the initiator and the oligonucleotide synthesized in pb is the expected size. Lane 3 of the agarose gel includes a polar solution including metal-polymer hybrid particles and initiator molecules to produce a number of framework compounds. Lane 4 of the agarose gel includes a solution having metal-polymer hybrid particles, initiator molecules, and an amount of a 0.4% by volume SDS solution. Lane 5 of the agarose gel includes metal-polymer hybrid particles comprised of iron oxide nanoparticles coated with PDMEAMA, initiator molecules, TdT enzymes, and a mixture of nucleoside triphosphates. Lane 5 is intended to show that oligonucleotides can be synthesized using the metal-polymer hybrid nanoparticles in an enzymatic oligonucleotide synthesis process. Lane 6 of the agarose gel includes metal-polymer hybrid particles, initiator molecules, TdT enzymes, a nucleotide mixture, and an amount of a 0.4% by volume SDS solution. Lane 6 is intended to show the release of oligonucleotides when exposed to the 0.4% by volume SDS solution where the oligonucleotides are produced using framework compounds in an enzymatic oligonucleotide synthesis process. Lanes 7-10 are meant to be control lanes that include a polar solution including metal-polymer hybrid nanoparticles particles in Lane 7 and a 0.4% by volume SDS solution in Lane 8. Lane 9 includes initiator molecules, TdT enzymes, and a nucleotide mixture and Lane 10 includes a solution including TdT enzymes and the nucleotide mixture.

### Example 3

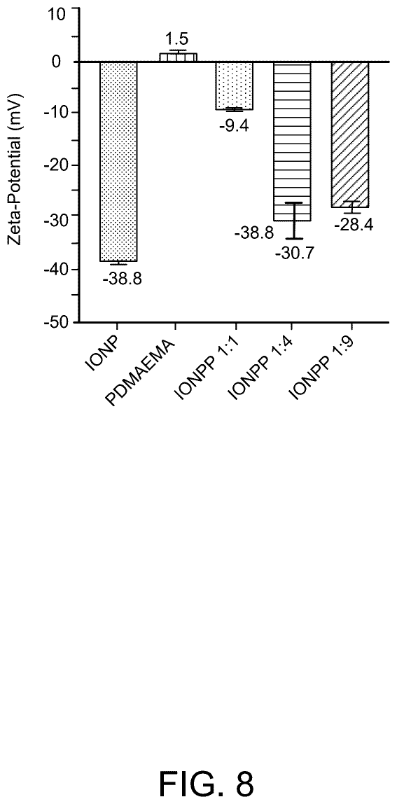

FIG. 8 shows zeta potential measurements for different combinations of amounts of a polymeric material and metallic particles.

### Example Aspects of the Disclosure

A numbered non-limiting list of examples of the present subject matter is presented below.

Example 1. An article comprises: a metallic particle; a polymeric layer disposed on the metallic particle, the polymeric layer being comprised of one or more polymeric materials; and a plurality of initiator molecules disposed on the polymeric layer, the plurality of initiator molecules comprising a plurality of nucleotides; wherein the metallic particle is bound to the polymeric layer by first electrostatic interactions between the metallic particle and one or more first functional groups of the one or more polymeric materials and the plurality of initiator molecules are bound to the polymeric layer by second electrostatic interactions between one or more second functional groups of the plurality of initiator molecules and the one or more first functional groups of the one or more polymeric materials.

In Example 2, the subject matter of example 1, wherein the metallic particle has magnetic properties.

In Example 3, the subject matter of example 2, wherein the metallic particle is a superparamagnetic metallic particle.

In Example 4, the subject matter of any one of examples 1-3, wherein the metallic particle comprises a metal oxide.

In Example 5, the subject matter of example 4, wherein the metallic particle comprises an iron oxide.

In Example 6, the subject matter of example 5, wherein the metallic particle comprises Fe₂O₃ or Fe₃₄.

In Example 7, the subject matter of any one of examples 1-6, having one or more first dimensions from about 100 nanometers to about 200 nanometers measured according to one or more dynamic light scattering techniques.

In Example 8, the subject matter of example 7, wherein the metallic particle has one or more second dimensions from about 80 nanometers to about 150 nanometers measured according to one or more dynamic light scattering techniques.

In Example 9, the subject matter of example 8, wherein the metallic particle and the article have a spherical shape and the one or more first dimensions of the article include a first diameter and the one or more second dimensions of the metallic particle include a second diameter.

In Example 10, the subject matter of any one of examples 1-9, wherein the one or more polymeric materials have a number average molecular weight from about 5 kilodaltons (kDa) to about 15 kDa.

In Example 11, the subject matter of example 10, wherein the one or more polymeric materials include at least one of Poly(N,N-dimethylaminoethyl methacrylate, polyethylenimine, poly-L-lysine, polyvinylamine, or polyallylamine.

In Example 12, the subject matter of any one of examples 1-11, wherein the polymeric layer has a thickness from about 10 nanometers to about 50 nanometers.

In Example 13, the subject matter of any one of examples 1-12, wherein the plurality of nucleotides of the plurality of initiator molecules include at least a portion of a nucleotide sequence of an M13 phage genome.

In Example 14, the subject matter of any one of examples 1-13, wherein the plurality of initiator molecules include from 2 nucleotides to 30 nucleotides.

In Example 15, the subject matter of any one of examples 1-14, wherein a ratio of a number of moles of the plurality of initiator molecules relative to a number of moles of the one or more polymeric materials is from about 3:1 to about 6:1.

In Example 16, the subject matter of any one of examples 1-15, wherein a ratio of a weight of the one or more polymeric materials of the polymeric layer relative to a weight of the plurality of initiator molecules can be from about 10:1 to about 18:1.

In Example 17, the subject matter of any one of examples 1-16, wherein a ratio of a weight of the one or more polymeric materials to a weight of an amount of metallic particles can be from about 3:1 to about 12:1.

In Example 18, the subject matter of any one of examples 1-17, wherein the one or more first functional groups of the one or more polymeric materials are positively charged in a polar solvent.

In Example 19, the subject matter of example 18, wherein the metallic particle forms a negatively charged ion in the polar solvent.

In Example 20, the subject matter of example 19, wherein the first electrostatic interactions comprise a first number of positively charged molecules of the one or more polymeric materials interacting with the negatively charged ion of the metallic particle.

In Example 21, the subject matter of example 20, wherein the one or more second functional groups of the plurality of initiator molecules are negatively charged in the polar solvent.

In Example 22, the subject matter of example 21, wherein the second electrostatic interactions comprise a second number of positively charged molecules of the one or more polymeric materials interacting with negatively charged molecules of the plurality of initiator molecules.

Example 23. A method comprises: combining an amount of metallic particles with an amount of one or more polymeric materials in one or more containers with an amount of a polar solvent to form coated metallic particles, wherein individual coated metallic particles comprise a polymeric layer of the one or more polymeric materials disposed on a metallic particle and the polymeric layer is bound to the metallic particle by first electrostatic interactions; and combining the coated metallic particles with a plurality of initiator molecules in the one or more containers to form a plurality of framework compounds, wherein the plurality of initiator molecules comprise a plurality of nucleotides and the plurality of initiator molecules are bound to the coated metallic particles by second electrostatic interactions.

In Example 24, the subject matter of example 23, wherein the polar solvent includes an aqueous solution.

In Example 25, the subject matter of example 23 or 24, comprises: applying a mixing device to combine the amount of metallic particles with the amount of one or more polymeric materials; and applying the mixing device to combine the coated metallic particles with the plurality of initiator molecules.

In Example 26, the subject matter of example 25, wherein the mixing device includes at least one of a mechanical stir bar, a paddle, or a sonicator.

In Example 27, the subject matter of example 25 or 26, wherein: the mixing device is applied to combine the amount of metallic particles with the amount of one or more polymeric materials for a first period of time from about 30 seconds to about 5 minutes; and the mixing device is applied to combine the amount of metallic particles with the amount of one or more polymeric materials for a second period of time from about 30 seconds to about 5 minutes.

In Example 28, the subject matter of any one of examples 23-27, wherein the coated metallic particles and the plurality of framework compounds are formed in the amount of polar solvent at temperatures from about 15° C. to about 30° C.

In Example 29, the subject matter of any one of examples 23-28, wherein a ratio of a weight of the amount of the one or more polymeric materials to a weight of the amount of the metallic particles is from about 3:1 to about 12:1.

In Example 30, the subject matter of any one of examples 23-29, wherein a ratio of a number of moles of the plurality of initiator molecules relative to a number of moles of the one or more polymeric materials in the polar solvent is from about 3:1 to about 6:1.

In Example 31, the subject matter of any one of examples 23-30, wherein: the metallic particles include negatively charged metallic particles in the polar solvent and the one or more polymeric materials comprise one or more positively charged functional groups in the polar solvent; and the first electrostatic interactions comprise the negatively charged metallic particles interacting with a first portion of the one or more positively charged functional groups of the one or more polymeric materials.

In Example 32, the subject matter of example 31, wherein the plurality of initiator molecules comprise one or more negatively charged functional groups in the polar solvent; and the second electrostatic interactions comprise the one or more negatively charged functional groups of the plurality of initiator molecules interacting with a second portion of the one or more positively charged functional groups of the one or more polymeric materials.

In Example 33, the subject matter of any one of examples 23-32, wherein the amount of metallic particles comprises a metal oxide.

In Example 34, the subject matter of example 33, wherein the amount of metallic particles comprise an iron oxide.

In Example 35, the subject matter of example 34, wherein the amount of metallic particle comprises Fe₂O₃ or Fe₃O₄.

In Example 36, the subject matter of any one of examples 23-35, wherein the one or more polymeric materials have a number average molecular weight from about 5 kilodaltons (kDa) to about 15 kDa.

In Example 37, the subject matter of any one of examples 23-36, wherein the one or more polymeric materials include at least one of Poly(N,N-dimethylaminoethyl methacrylate, polyethylenimine, poly-L-lysine, polyvinylamine, or polyallylamine.

In Example 38, the subject matter of any one of examples 23-37, wherein the plurality of nucleotides of the plurality of initiator molecules include at least a portion of a nucleotide sequence of an M13 phage genome.

In Example 39, the subject matter of any one of examples 23-38, wherein the plurality of initiator molecules include from 2 nucleotides to 30 nucleotides.

In Example 40, the subject matter of any one of examples 23-39, comprises: adding a number of nucleotides to individual initiator molecules of the plurality of initiator molecules to produce a plurality of oligonucleotides bound to the polymeric layer; and adding a separation solution including an amount of a surfactant to the one or more containers to separate the plurality of oligonucleotides from the polymeric layer.

In Example 41, the subject matter of example 40, comprises: obtaining an amount of digital data; and determining nucleotide sequences to encode the amount of digital data; wherein the number of nucleotides added to the individual initiator molecules of the plurality of initiator molecules to produce the plurality of oligonucleotides correspond to at least a portion of the nucleotide sequences.

In Example 42, the subject matter of example 40 or 41, wherein the separation solution comprises at least about 0.03% by volume of sodium dodecyl sulfate.

In Example 43, the subject matter of example 41 or 42, comprises: performing one or more sequencing operations with respect to at least a portion of the plurality of oligonucleotides to determine nucleotide sequences of the at least a portion of the plurality of oligonucleotides; and analyzing the nucleotide sequences according to a decoding scheme to determine one or more portions of the digital data that correspond to the at least a portion of the plurality of oligonucleotides.

Example 44. A method comprises: providing a plurality of framework compounds in a polar solvent disposed in one or more containers, the plurality of framework compounds comprising a number of coated metallic particles having a plurality of initiator molecules bound to the number of coated metallic particles, wherein individual coated metallic particles comprise a metallic particle coated with a polymeric layer that includes one or more polymeric materials; adding a number of nucleotides to individual initiator molecules of the plurality of initiator molecules to produce a plurality of oligonucleotides bound to the polymeric layer; and adding a rinsing solution including an amount of a surfactant to the one or more containers to separate the plurality of oligonucleotides from the polymeric layer.

In Example 45, the subject matter of example 44, wherein the rinsing solution includes an additional polar solvent and the amount of surfactant included in the rinsing solution is from about 0.02% by volume to about 1% by volume of a total volume of the rinsing solution.

In Example 46, the subject matter of example 44 or 45, wherein the surfactant includes sodium dodecyl sulfate.

In Example 47, the subject matter of any one of examples 44-46, comprises: obtaining an amount of digital data; and determining nucleotide sequences to encode the amount of digital data; wherein the number of nucleotides added to the individual initiator molecules of the plurality of initiator molecules to produce the plurality of oligonucleotides correspond to at least a portion of the nucleotide sequences.

In Example 48, the subject matter of example 47, wherein the number of nucleotides are added to the individual initiator molecules by providing a plurality of deoxynucleoside triphosphate (dNTP) solutions to the one or more containers, individual dNTP solutions comprising an aqueous solution including an amount of an individual nucleotide.

In Example 49, the subject matter of example 48, wherein the number of nucleotides are added to the individual initiator molecules by providing one or more enzymes to the one or more containers in conjunction with the plurality of dNTP solutions.

In Example 50, the subject matter of example 49, wherein the plurality of dNTP solutions include a first dNTP solution comprising an amount of deoxyadenosine triphosphate, a second dNTP solution comprising an amount of deoxythymidine triphosphate, a third dNTP solution comprising an amount of deoxyguanosine triphosphate, and a fourth dNTP solution comprising an amount of deoxycytidine triphosphate.

In Example 51, the subject matter of example 50, comprises: determining a first nucleotide to be added to at least a portion of the plurality of initiator molecules according to a nucleotide sequence encoding a segment of the amount of the digital data; adding a first dNTP solution to the one or more containers, the first dNTP solution including a first deoxynucleoside triphosphate that corresponds to the first nucleotide; adding a first amount of the one or more enzymes to the one or more containers, such that the first dNTP solution and the one or more enzymes are disposed in the one or more containers concurrently; and producing first intermediate framework compounds bound to the polymeric layer, the first intermediate framework compounds including one or more first instances of the first nucleotide added to the at least a portion of the plurality of initiator molecules.

In Example 52, the subject matter of example 51, comprises: determining a second nucleotide to add to at least a portion of the first intermediate framework compounds according to the nucleotide sequence encoding the segment of the amount of the digital data; adding a second dNTP solution to the one or more containers, the second dNTP solution including a second deoxynucleoside triphosphate that corresponds to the second nucleotide; adding a second amount of the one or more enzymes to the one or more containers, such that the second dNTP solution and the one or more enzymes are disposed in the one or more containers concurrently; and producing second intermediate framework compounds bound to the polymeric layer, the second intermediate framework compounds including one or more second instances of the second nucleotide added to the one or more first instances of the first nucleotide of the at least a portion of the first intermediate framework compounds.

In Example 53, the subject matter of example 52, wherein the first dNTP solution and the first amount of the one or more enzymes are disposed concurrently in the one or more containers for a period of time to produce the first intermediate framework compounds; and the method comprising: subsequent to the period of time, applying a magnetic field to the one or more containers to cause the first intermediate framework compounds to be bound to one or more surfaces of the one or more containers; and removing, using one or more washing solutions, a remainder of the first dNTP solution and a remainder of the first amount of the one or more enzymes from the one or more containers while the magnetic field is applied to the one or more containers.

In Example 54, the subject matter of example 53, wherein the second dNTP solution and the second amount of the one or more enzymes are added to the one or more containers after removal of the remainder of the first dNTP solution and the remainder of the first amount of the one or more enzymes.

In Example 55, the subject matter of any one of examples 52-54, wherein the one or more first instances of the first nucleotide comprise a first homopolymer including from 1 to 10 instances of the first nucleotide and the one or more second instances of the second nucleotide comprise a second homopolymer including from 1 to 10 instances of the second nucleotide.

In Example 56, the subject matter of example 49-55, wherein the one or more enzymes include terminal deoxynucleotidyl transferase (TdT).

In Example 57, the subject matter of any one of examples 47-56, comprises: performing one or more sequencing operations with respect to the plurality of oligonucleotides to generate sequencing data, the sequencing data including sequencing reads that correspond to nucleic acid sequences of the plurality of oligonucleotides; and analyzing the sequencing data in relation to one or more decoding schema to determine one or more segments of the amount of digital data that correspond to the nucleic acid sequences of the sequencing reads.

In Example 58, the subject matter of example 57, wherein the one or more sequencing operations are performed in response to receiving a request to retrieve one or more portions of the amount of digital data.

In Example 59, the subject matter of example 58, comprises causing at least a portion of the one or more portions of the amount of digital data to at least one of (i) be displayed by a display device or (ii) be accessible to one or more applications being executed by one or more computing devices.

Example 60. A formulation comprises: a polar solvent; and a plurality of framework compounds, individual framework compounds of the plurality of framework compounds corresponding to any of the articles of claims 1-22.

In Example 61, the subject matter of example 60, wherein the polar solvent includes H2O.

In Example 62, the subject matter of example 60 or 61, wherein a concentration of the plurality of framework compounds in the polar solvent is from about 10 milligrams (mg) to 30 mg of the plurality of framework compounds to 1 milliliter (mL) of the polar solvent.

What is claimed is:

1. An article comprising:
a metallic particle;
a polymeric layer disposed on the metallic particle, the polymeric layer being comprised of one or more polymeric materials; and
a plurality of initiator molecules disposed on the polymeric layer, the plurality of initiator molecules comprising a plurality of nucleotides;
wherein the metallic particle is bound to the polymeric layer by first electrostatic interactions between the metallic particle and one or more first functional groups of the one or more polymeric materials and the plurality of initiator molecules are bound to the polymeric layer by second electrostatic interactions between one or more second functional groups of the plurality of initiator molecules and the one or more first functional groups of the one or more polymeric materials.

2. The article of claim 1, wherein the metallic particle comprises Fe₂O₃ or Fe₃O₄.

3. The article of claim 1, wherein the metallic particle has a spherical shape having a diameter from about 80 nanometers to about 150 nanometers measured according to one or more dynamic light scattering techniques.

4. The article of claim 1, wherein the one or more polymeric materials have a number average molecular weight from about 5 kilodaltons (kDa) to about 15 kDa.

5. The article of claim 4, wherein the one or more polymeric materials include at least one of Poly(N,N-dimethylaminoethyl methacrylate, polyethylenimine, poly-L-lysine, polyvinylamine, or polyallylamine.

6. The article of claim 1, wherein the polymeric layer has a thickness from about 10 nanometers to about 50 nanometers.

7. The article of claim 1, wherein the plurality of initiator molecules include from 2 nucleotides to 30 nucleotides.

8. The article of claim 1, wherein:
a ratio of a weight of the one or more polymeric materials of the polymeric layer relative to a weight of the plurality of initiator molecules can be from about 10:1 to about 18:1; and
a ratio of a weight of the one or more polymeric materials to a weight of an amount of metallic particles can be from about 3:1 to about 12:1.

9. The article of claim 1, wherein:
the first electrostatic interactions comprise a first number of positively charged molecules of the one or more polymeric materials interacting with a negatively charged ion of the metallic particle; and
the second electrostatic interactions comprise a second number of positively charged molecules of the one or more polymeric materials interacting with negatively charged molecules of the plurality of initiator molecules.

10. A method comprising:
combining an amount of metallic particles with an amount of one or more polymeric materials in one or more containers with an amount of a polar solvent to form coated metallic particles, wherein individual coated metallic particles comprise a polymeric layer of the one or more polymeric materials disposed on a metallic particle and the polymeric layer is bound to the metallic particle by first electrostatic interactions; and
combining the coated metallic particles with a plurality of initiator molecules in the one or more containers to form a plurality of framework compounds, wherein the plurality of initiator molecules comprise a plurality of nucleotides and the plurality of initiator molecules are bound to the coated metallic particles by second electrostatic interactions.

11. The method of claim 10, wherein the coated metallic particles and the plurality of framework compounds are formed in the amount of polar solvent at temperatures from about 15° C. to about 30° C.

12. The method of claim 10, wherein:
the metallic particles include negatively charged metallic particles in the polar solvent and the one or more polymeric materials comprise one or more positively charged functional groups in the polar solvent;
the first electrostatic interactions comprise the negatively charged metallic particles interacting with a first portion of the one or more positively charged functional groups of the one or more polymeric materials;
the plurality of initiator molecules comprise one or more negatively charged functional groups in the polar solvent; and
the second electrostatic interactions comprise the one or more negatively charged functional groups of the plurality of initiator molecules interacting with a second portion of the one or more positively charged functional groups of the one or more polymeric materials.

13. The method of claim 10, comprising:
adding a number of nucleotides to individual initiator molecules of the plurality of initiator molecules to produce a plurality of oligonucleotides bound to the polymeric layer; and
adding a separation solution including an amount of a surfactant to the one or more containers to separate the plurality of oligonucleotides from the polymeric layer.

14. The method of claim 13, comprising:
obtaining an amount of digital data; and
determining nucleotide sequences to encode the amount of digital data;
wherein the number of nucleotides added to the individual initiator molecules of the plurality of initiator molecules to produce the plurality of oligonucleotides correspond to at least a portion of the nucleotide sequences.

15. The method of claim 13, wherein the separation solution comprises at least about 0.03% by volume of sodium dodecyl sulfate.

16. The method of claim 14, comprising:
performing one or more sequencing operations with respect to at least a portion of the plurality of oligonucleotides to determine nucleotide sequences of the at least a portion of the plurality of oligonucleotides, wherein the one or more sequencing operations are performed in response to receiving a request to retrieve one or more portions of the amount of digital data;
analyzing the nucleotide sequences according to a decoding scheme to determine one or more portions of the digital data that correspond to the at least a portion of the plurality of oligonucleotides; and
causing at least a portion of the one or more portions of the amount of digital data to at least one of (i) be displayed by a display device or (ii) be accessible to one or more applications being executed by one or more computing devices.

17. A method comprising:
providing a plurality of framework compounds in a polar solvent disposed in one or more containers, the plurality of framework compounds comprising a number of coated metallic particles having a plurality of initiator molecules bound to the number of coated metallic particles, wherein individual coated metallic particles comprise a metallic particle coated with a polymeric layer that includes one or more polymeric materials;
adding a number of nucleotides to individual initiator molecules of the plurality of initiator molecules to produce a plurality of oligonucleotides bound to the polymeric layer; and
adding a rinsing solution including an amount of a surfactant to the one or more containers to separate the plurality of oligonucleotides from the polymeric layer.

18. The method of claim 17, comprising:
obtaining an amount of digital data; and
determining nucleotide sequences to encode the amount of digital data;
wherein:
the number of nucleotides added to the individual initiator molecules of the plurality of initiator molecules to produce the plurality of oligonucleotides correspond to at least a portion of the nucleotide sequences;
the number of nucleotides are added to the individual initiator molecules by providing a plurality of deoxynucleoside triphosphate (dNTP) solutions to the one or more containers, individual dNTP solutions comprising an aqueous solution including an amount of an individual nucleotide; and
the number of nucleotides are added to the individual initiator molecules by providing one or more enzymes to the one or more containers in conjunction with the plurality of dNTP solutions.

19. The method of claim 18, comprising:
determining a nucleotide to be added to at least a portion of the plurality of initiator molecules according to a nucleotide sequence encoding a segment of the amount of the digital data;
adding a dNTP solution to the one or more containers, the dNTP solution including a deoxynucleoside triphosphate that corresponds to the nucleotide;
adding an amount of the one or more enzymes to the one or more containers, such that the dNTP solution and the one or more enzymes are disposed in the one or more containers concurrently; and
producing intermediate framework compounds bound to the polymeric layer, the intermediate framework compounds including one or more instances of the nucleotide added to the at least a portion of the plurality of initiator molecules.

20. The method of claim 19, wherein the dNTP solution and the amount of the one or more enzymes are disposed concurrently in the one or more containers for a period of time to produce the intermediate framework compounds; and the method comprising:
subsequent to the period of time, applying a magnetic field to the one or more containers to cause the intermediate framework compounds to be bound to one or more surfaces of the one or more containers; and
removing, using one or more washing solutions, a remainder of the dNTP solution and a remainder of the amount of the one or more enzymes from the one or more containers while the magnetic field is applied to the one or more containers.
